(19) United States
(12) Patent Application Publication
(10) Pub. No.: US 2006/0166906 A1
(43) Pub. Date: Jul. 27, 2006

(54) 6-O-substituted erythromycin derivatives having improved gastrointestinal tolerance

(76) Inventors: Zhenkun Ma, Gurnee, IL (US); Ly Tam Phan, Malden, MA (US); Richard F. Clark, Gurnee, IL (US); Suoming Zhang, Brandford, CT (US); Stevan Djuric, Libertyville, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 09/978,990

(22) Filed: Oct. 16, 2001

(51) Int. Cl. A61K 31/7052; C07H 17/08

(52) U.S. Cl. 514/29; 536/7.4; 536/17.4

(57) ABSTRACT

Compounds having formula (I) 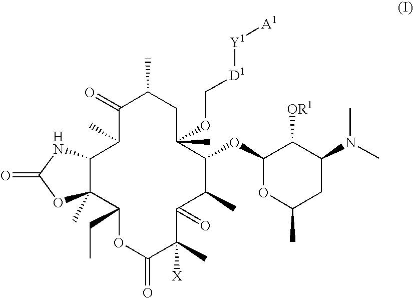 are useful for treating bacterial infections while avoiding the concomitant liability of gastrointestinal intolerance. Compositions containing the compounds and methods of treatment using the compounds are also disclosed.

### THIS APPLICATION CLAIMS PRIORITY TO CO-PENDING U.S. PROVISIONAL APPLICATION NO.60/240,777 FILED OCT. 16,2000

### TECHNICAL FIELD

This invention relates to novel semi-synthetic erythromycin derivatives having antibacterial activity, compositions containing the compounds, and methods of treatment using the compounds. These compounds have a lower incidence of GI irritation than the erythromycin derivatives of the prior art.

### BACKGROUND OF THE INVENTION

The escalation of resistance to antibiotics once useful for treatment of bacterial infections resulting from pathogens such as Staphylococcus aureus is problematic in the United States and Europe (Drugs Exp. Clin. Res., XX, 215-224 (1994); Am. J. Surg., 5A (Suppl.), 8S-12S (1995); Drugs, 48, 678-688 (1994); and Current Pharmaceutical Design, 2(2), 175-194 (1996)). Thus, the development of new broad spectrum synthetic and semi-synthetic antibacterial compounds is the subject of constant current research.

Reference is made to commonly owned U.S. Pat. No. 5,866,549 and PCT application WO/21871, published May 6, 1999, each of which teachs 6-O-substituted ketolide antibacterial compounds; although neither reference teaches by specific example combinations wherein the 6-O-substituted group is lower alkenyl or lower alkynyl wherein the lower alkynyl or lower alkenyl is substituted with an isoxazole, oxazole, or isothiazole substituent. While the compounds described in these applications represent an advance in antibacterial therapy, they suffer from the side effect of gastrointestinal intolerance typically associated with erythromycins (cf. Pilot and Williams, Macrolides: Chemistry, Pharmacology and Clinical Uses; Briskier, Neu, and Tulkens, Eds.; Blackwell: Paris, 1993; pp. 659-673; and Itoh et al., Antimicrob. Agents Chemother., 26, 863-869 (1984)).

Thus, erythromycin derivatives which produce a lower incidence of gastrointestinal intolerance and the side effects associated therewith, such as nausea and vomiting, would represent an important contribution to the art.

### SUMMARY OF THE INVENTION

In its principle embodiment, therefore, the instant invention provides a series of 6-O-substituted ketolides with an unexpectedly improved gastrointestinal tolerability profile, the ketolides having structural formula (I) 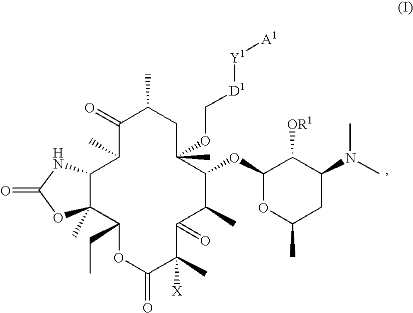
or a therapeutically acceptable salt or prodrug thereof, wherein

X is selected from hydrogen and fluoride;

D¹ is selected from CH═CH or C≡C;

Y¹ is selected from isoxazole, oxazole, isothiazole, dihydroisoxazole, and dihydro-oxazole;

A¹ is selected from aryl and heteroaryl; and

R¹ is selected from hydrogen and R^{P} wherein R^{p} is a hydroxyl protecting group.

In another embodiment, the invention provides any compound, including metabolic precursors of the inhibitor compounds, which contain an essential inhibitory group as disclosed herein. These inhibitory groups can be in masked form or in therapeutically effective prodrug form and can be converted or released by metabolic or other processes after administration to a patient.

In yet another embodiment, the invention provides compositions comprising the compounds in combination with a therapeutically acceptable excipient.

In still yet another embodiment, the invention provides a method of treating bacterial infections the method comprising administering a therapeutically effective amount of a compound
having structural formula (I) 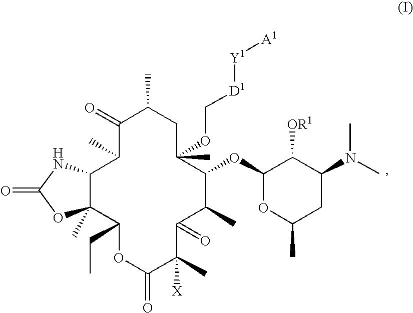
or therapeutically acceptable salt or prodrug thereof, wherein

X is selected from hydrogen and fluoride;

D¹ is selected from CH═CH or C≡C;

Y¹ is selected from isoxazole, oxazole, isothiazole, dihydroisoxazole, and dihydro-oxazole;

A¹ is selected from aryl and heteroaryl; and

R¹ is selected from hydrogen and R^{p} wherein R^{p} is a hydroxyl protecting group.

In still yet another embodiment, the invention provides a method for the preparation of the compounds of formula (Ia) 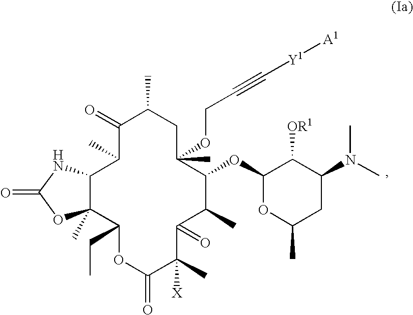
or a therapeutically acceptable salt or prodrug thereof, wherein

X is selected from hydrogen and fluoride;

Y¹ is selected from isoxazole, oxazole, isothiazole, and isothiazole;

A¹ is selected from aryl and heteroaryl; and

R¹ is selected from hydrogen and R^{p} wherein R^{p} is a hydroxyl protecting group, the method comprising:

(a) reacting a compound of formula (vi) 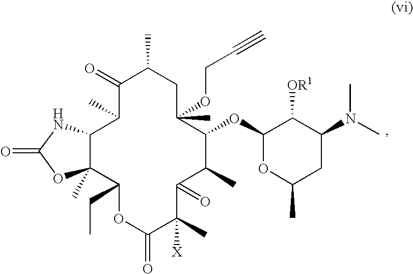
wherein
X is selected from hydrogen and fluoride, and
R¹ is selected from hydrogen and R^{p} wherein R^{p} is a hydroxyl protecting group,

with a compound of formula (vii)
I—Y¹A¹ (vii),
wherein
Y¹ is selected from isoxazole, oxazole, and isothiazole, and
A¹ is selected from aryl and heteroaryl,

a base, a coupling catalyst, and, optionally, an additive; and

(b) optionally deprotecting the product of step (a).

In still yet another embodiment, the invention provides a method for the preparation of the compounds of formula (Ib) 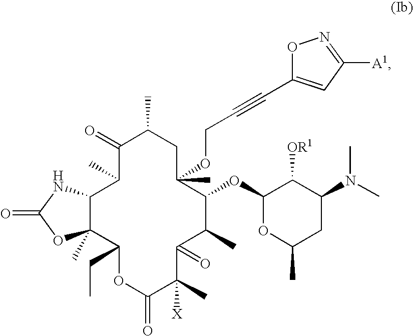
or a therapeutically acceptable salt or prodrug thereof, wherein

X is selected from hydrogen and fluoride;

A¹ is selected from aryl and heteroaryl; and

R¹ is selected from hydrogen and R^{p} wherein R^{p} is a hydroxyl protecting group, the method comprising:

(a) reacting a compound of formula (x) 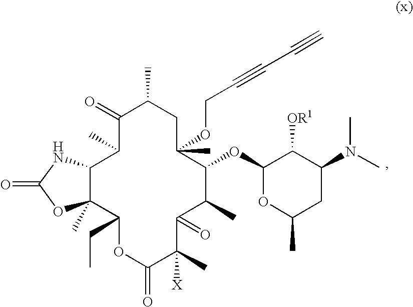
wherein
X is selected from hydrogen and fluoride, and
R¹ is selected from hydrogen and R^{p} wherein R^{p} is a hydroxyl protecting group,
   with a compound of formula (ii) 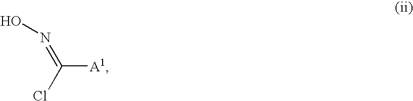
   wherein
A¹ is selected from aryl and heteroaryl,

and a base;

and

(b) optionally deprotecting the product of step (a).

In a preferred embodiment of the compound of formula (x),

X and R¹ are hydrogen;

X is hydrogen and R¹ is R^{p} wherein R^{p} is acetyl;

X is hydrogen and R¹ is R^{p} wherein R^{p} is benzoyl;

X is fluoride and R¹ is hydrogen;.

X is fluoride and R¹ is R^{p} wherein R^{p} is acetyl; and

X is fluoride and R¹ is R^{p} wherein R^{p} is benzoyl.

### DETAILED DESCRIPTION OF THE INVENTION

The instant compounds are substituted ketolide antibiotics of formula (I) 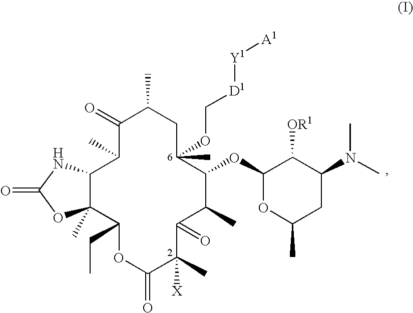
which have been numbered at the C-2 and C-6 positions for illustrative purposes. The compounds contain a number of asymmetric centers and optional substitution of the hydrogen atom at C-2 by a fluorine atom. Each variable substituent at C-2 is represented by X. In a preferred embodiment for the practice of the invention, X is hydrogen or fluoride. In a particularly preferred embodiment, X is fluoride.

D¹ can also vary without departing from the intent of the invention and can be C₂-alkynylene or C₂-alkenylene, the latter of which provides geometric isomers of the compounds. The invention contemplates the various geometric isomers and mixtures thereof which result from the disposal of substituents around a carbon-carbon double bond. Substituents around a carbon-carbon double bond are designated as being of Z or E configuration, wherein the term “Z” refers to higher order substituents on the same side of the carbon-carbon double bond, and the term “E” refers to higher order substituents on opposite sides of the carbon-carbon double bond. A thorough discussion of E and Z isomerism is provided in J. March, Advanced Organic Chemistry. Reactions, Mechanisms, and Structure, 4th ed., John Wiley & Sons, New York, 1992, pp. 109-112. Accordingly, it will be appreciated by a skilled practioner that compounds of formula (Ia) 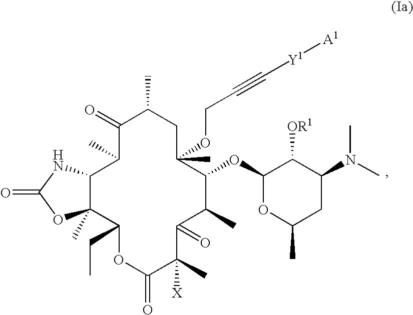
compounds of formula Z-(Ic) 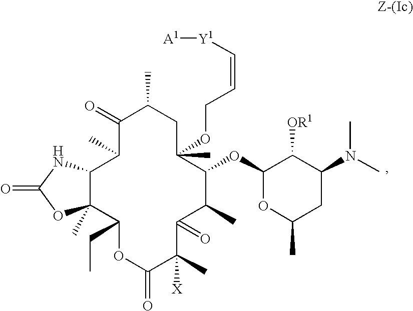
and compounds of formula E-(Ic) 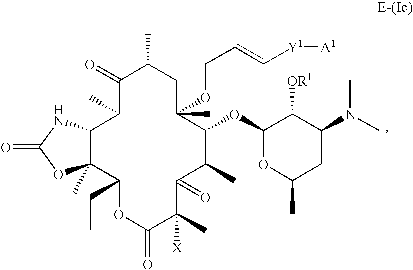
and therapeutically acceptable salts or prodrugs thereof, are contemplated as being within the scope of the invention. In a preferred embodiment for the practice of the invention, D¹ is C₂-alkynylene, as exemplified by compounds of formula (Ia).

The compounds further comprise substituted heteroarylene or heterocyclene rings, represented by Y¹, connected to the parent molecular group through groups represented by D¹ and substituted by groups represented by A¹. The groups represented by Y¹ are stable, 5-membered, diradical rings containing one nitrogen atom, one atom selected from oxygen and sulfur, and the remaining atoms are carbon. The rings are connected to D¹ and are substituted by A¹ through substitutable carbons. For combinations within Y¹ which contain nitrogen and oxygen atoms, the heteroatoms, i.e. non-carbon atoms, can be in adjacent or non-adjacent positions. For combinations within Y¹ which contain nitrogen and sulfur atoms, the heteroatoms are in adjacent positions. In each of the aformentioned cases, the rings can contain one or two double bonds. In a preferred embodiment for the practice of the invention, Y¹ is a five membered ring with two double bonds and a nitrogen and oxygen atom in adjacent positions, i.e. isoxazole, the structure and atom numbering of which is shown directly below for illustrative purposes.

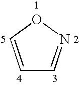

In a particularly preferred embodiment, the isoxazole ring is substituted by A¹ and D¹ on the C-3 and C-5 positions, respectively, to provide a isoxazol-3,5-diyl. Accordingly, taking the listing of preferred substituents and combinations thereof, it will be appreciated by a skilled practioner that compounds of formula (Ib) 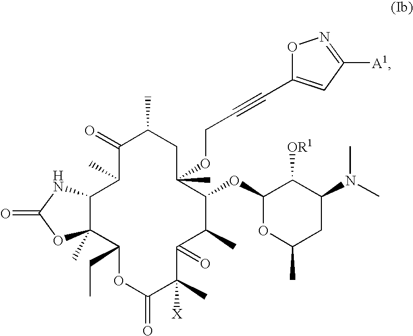
and therapeutically acceptable salts or prodrugs thereof, are contemplated as being within the scope of the invention.

A¹ can also vary considerably without departing from the intent of the invention and can be aryl or heteroaryl. Preferred embodiments of A¹ include unsubstituted or substituted monocyclic, aromatic groups such as phenyl, pyridyl, pyrimidinyl, thienyl, thiazolyl, tetrazolyl, and the like, and unsubstituted or substituted bicyclic, aromatic groups such as quinolinyl, benzothienyl, imidazo(2,1-b)thiazolyl, and the like. Each of the aformentioned groups, represented by A¹, are connected to Y¹ through substitutable carbon atoms in the ring. Thus, Y¹ substituents such as, for instance, and by way of example only, pyrid-2-yl, pyrid-3-yl, pyrid-4-yl, pyrimidin-2-yl, pyrimidin-4-yl, pyrimidin-5-yl, and the like, are contemplated as being within the scope of the invention. A preferred embodiment for the practice of the invention is unsubstituted pyridyl, and a particularly preferred embodiment is unsubstituted pyrid-2-yl. Accordingly, taking the listing of preferred substituents and combinations thereof, it will be appreciated by a skilled practioner that compounds of formula (Ii) 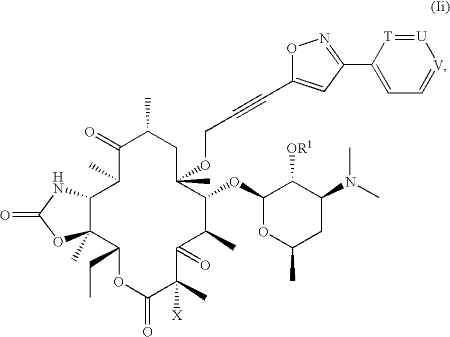
and therapeutically acceptable salts or prodrugs thereof, wherein one of T, U, and V is nitrogen and the remainder are carbon; and, more specifically, compounds of formula (Ii) wherein T is nitrogen and U and V are each carbon, are contemplated as being within the scope of the invention.

It is believed that when the compounds have attached thereto a hydroxyl, amino, or carboxylic acid group, prodrugs can be prepared from the compounds by attaching thereto a prodrug-forming group such as, but not limited to, include carboxyl, hydroxyl, and amino protecting groups. These prodrugs can then be rapidly transformed in vivo to the parent compound, such as, for example, by hydrolysis in blood. The term “therapeutically acceptable prodrug,” means those prodrugs of the compounds which are suitable for use in contact with the tissues of humans and lower animals with undue toxicity, irritation, allergic response, and the like, commensurate with a reasonable benefit/risk ratio, and effective for their intended use, as well as the zwitterionic forms, wherein possible, of the compounds.

As used in the specification, the following terms have the meanings assigned:

The term “additive,” means monodentate phosphorus-containing ligands of formulas P(R^{c})₃ (phosphines), P(OR^{d})₃ (phosphites) and As(R^{c})₃ (arsines), wherein each R^{c} is independently hydrogen; alkyl such as methyl, ethyl, and tert-butyl; cycloalkyl such as cyclopropyl and cyclohexyl; optionally substituted aryl such as phenyl, naphthyl, and ortho-tolyl; and optionally substituted heteroaryl such as furyl and pyridyl; and wherein each R^{d} is independently alkyl such as methyl, ethyl, and tert-butyl; cycloalkyl such as cyclopropyl and cyclohexyl; optionally substituted aryl such as phenyl, naphthyl, and ortho-tolyl; and optionally substituted heteroaryl such as furyl and pyridyl. Specific examples of these additives include tri(alkyl)phosphines such as trimethylphosphine, triethylphosphine, tributylphosphine, and the like; tri(cycloalkyl)phosphines such as tricyclopropylphosphine, tricyclohexylphosphine, and the like; tri(aryl)phosphines such as triphenylphosphine, trinaphthylphosphine, and the like; tri(heteroaryl)phosphines such as tri(fury-2-yl)phosphine, tri(pyrid-3-yl)phosphine, and the like; tri(alkyl)phosphites such as trimethylphosphite, triethylphosphite, tributylphosphite, and the like; tri(cycloalkyl)-phosphites such as tricyclopropylphosphite, tricyclohexylphosphite, and the like; tri(aryl)phosphites such as triphenylphosphite, trinaphthylphosphite, and the like; tri(heteroaryl)phosphites such as tri(fury-2-yl)phosphite, tri(pyrid-3-yl)phosphite, and the like; and triphenylarsine, and the like. The term “additive,” also means bidentate phosphines such as 1,4-bis(diphenylphosphino)butane (dppb), 1,2-bis(diphenyl-phosphino)ethane (dppe), 1,1-bis(diphenylphosphino)methane (dppm), 1,2-bis(dimethyl-phosphino)ethane (dmpe), 1,1′-bis(diphenylphosphino)ferrocene (dppf), and the like. The term “additive,” also means copper salts such as copper(I) iodide and copper(I) chloride.

The term “alkanoyl,” means an alkyl group attached to the parent molecular group through a carbonyl group.

The term “alkanoyloxy,” means an alkanoyl group attached to the parent molecular group through an oxygen atom.

The term “alkoxy,” means an alkyl group attached to the parent molecular group through an oxygen atom.

The term “alkoxycarbonyl,” means an alkoxy group attached to the parent molecular group through a carbonyl group.

The term “alkoxyalkoxy,” means an alkoxy group to which attached at least one other alkoxy group.

The term “alkyl,” means a straight or branched chain saturated hydrocarbon radical having from one to six carbon atoms.

The term “alkenyl,” means a straight or branched chain hydrocarbon radical having from two to six carbon atoms and at least one carbon-carbon double bond.

The term “C₂-alkenylene,” means a diradical formed by the removal of a hydrogen atom from each carbon atom of ethylene.

The term “alkynyl,” means a straight or branched chain hydrocarbon radical having from two to six carbon atoms and at least one carbon-carbon triple bond.

The term “C₂-alkynylene,” means a diradical formed by the removal of both hydrogen atoms from acetylene.

The term “amino,” means -NH₂ or derivatives thereof formed by independent replacement of one or both hydrogen atoms thereon with a substituent or substituents independently selected from alkyl, alkanoyl, aryl, arylalkyl, cycloalkyl, cycloalkylalkyl, heteroaryl, heteroarylalkyl, and an amino protecting group.

The term “aminoalkyl,” means an alkyl group, as defined herein, to which is attached at least one amino substituent.

The terms “amino protecting group,” or “nitrogen protecting group,” mean selectively introducible and removable groups which protect amino groups against undesirable side reactions during synthetic procedures. Examples of amino protecting groups include methoxycarbonyl, ethoxycarbonyl, trichloroethoxycarbonyl, benzyloxycarbonyl (Cbz), chloroacetyl, trifluoroacetyl, phenylacetyl, benzoyl (Bn), benzyl (Bz), dimethoxybenzyl, tert-butoxycarbonyl (Boc), para-methoxybenzyloxycarbonyl, isopropoxycarbonyl, phthaloyl, succinyl, diphenylmethyl, triphenylmethyl (trityl), methanesulfonyl, para-toluenesulfonyl, trimethylsilyl, triethylsilyl, triphenylsilyl, and the like. Amino protecting groups can also be used as prodrug-forming groups.

The term “aryl,” means an aromatic, carbocyclic ring containing six carbon atoms. The aryl group can be optionally fused to another aryl group, a cycloalkyl group, or a cycloalkenyl group. Aryl groups of the invention are exemplified by phenyl, naphthyl, indenyl, indanyl, dihydronaphthyl, tetrahydronaphthyl, and the like. The aryl groups are connected to the parent molecular group through a substitutable carbon. The aryl groups of the invention can be optionally substituted with 1-5 substituents independently selected from alkyl, alkenyl, alkynyl, alkoxyalkoxy, amino, aminoalkyl, cyano, cyanoalkyl, halo, haloalkyl, nitro, perfluoroalkyl, perfluoroalkoxy, oxo, —(CH₂)ₐC(O)R⁵, —(CH₂)ₐC(O)OR⁵, —(CH₂)ₐN(R⁵)C(O)R⁵, —(CH₂)ₐC(O)N(R⁵)₂, —(CH₂)ₐN(R⁵)C(O)N(R⁵)₂, —(CH₂)ₐOR⁵, —(CH₂)ₐSO₂R⁵, (CH₂)ₐSR⁶, and —(CH₂)ₐR⁷;

wherein a is zero to six;

R⁵ is selected from hydrogen, unsubstituted or substituted alkyl, unsubstituted or substituted cycloalkyl, unsubstituted or substituted aryl, unsubstituted or substituted heteroaryl, and unsubstituted or substituted heterocyclyl; R⁶ is selected from unsubstituted or substituted alkyl, unsubstituted or substituted cycloalkyl, unsubstituted or substituted aryl, unsubstituted or substituted heteroaryl, and unsubstituted or substituted heterocyclyl; and R⁷ is selected from unsubstituted or substituted aryl, unsubstituted or substituted heteroaryl, and unsubstituted or substituted heterocyclyl;

the term “substituted alkyl” means an alkyl group substituted with 1-3 substituents independently selected from alkoxy, alkanoyloxy, alkoxycarbonyl, amino, unsubstituted phenyl, carboxamido, carboxy, cyano, unsubstituted cycloalkyl, halo, unsubstituted heteroaryl, hydroxy, nitro, perfluoroalkyl, oxo, and thioalkoxy.

the term “substituted aryl” means an aryl substituted with 1-5 substituents independently selected from unsubstituted alkyl, alkenyl, alkynyl, alkoxy, alkanoyloxy, alkoxycarbonyl, amino, carboxamido, carboxy, cyano, cycloalkyl, halo, hydroxy, nitro, perfluoroalkyl, oxo, and thioalkoxy.

the term “substituted cycloalkyl” means a cycloalkyl group substituted with one to four substituents independently selected from unsubstituted alkyl, alkoxy, alkanoyloxy, alkoxycarbonyl, amino, unsubstituted phenyl, carboxamido, carboxy, cyano, halo, hydroxy, nitro, perfluoroalkyl, oxo, and thioalkoxy.

the term “substituted heteroaryl” means a heteroaryl substituted with one to four substituents independently selected from unsubstituted alkyl, alkenyl, alkoxy, alkanoyloxy, alkoxycarbonyl, amino, carboxamido, carboxy, cyano, cycloalkyl, halo, hydroxy, nitro, perfluoroalkyl, oxo, and thioalkoxy.

the term “substituted heterocyclyl” means a heterocyclyl group substituted with one to four substituents independently selected from unsubstituted alkyl, alkenyl, alkoxy, alkanoyloxy, alkoxycarbonyl, amino, unsubstituted phenyl, carboxamido, carboxy, cyano, halo, hydroxy, nitro, perfluoroalkyl, oxo, and thioalkoxy.

The term “arylalkyl,” means an alkyl group to which is attached at least one aryl group.

The term “base,” means reagents capable of accepting protons during the course of a chemical reaction. Examples of bases include carbonates such as lithium carbonate, lithium bicarbonate, sodium carbonate, sodium bicarbonate, potassium carbonate, potassium bicarbonate, cesium carbonate, and the like; phosphates such as potassium phosphate, potassium hydrogen phosphate, potassium dihydrogen phosphate, and the like; trialkylamines such as triethylamine, diisopropylethylamine, N,N,N,N-tetramethyl-1,8-naphthalenediamine (Proton-Sponge®), and the like; heterocyclic amines such as imidazole, pyridine, pyridazine, pyrimidine, pyrazine, and the like; and bicyclic amines such as 1,5-diazabicyclo[4.3.0]non-5-ene (DBN), 1,8-diazabicyclo[5.4.0]undec-7-ene (DBU), and the like. The base chosen for a particular conversion depends on the nature of the starting materials, the solvent or solvents in which the reaction is conducted, and the temperature at which the reaction is conducted.

The term “carbonyl,” means —C(O)—.

The term “carboxy,” means —CO₂H.

The term “carboxy protecting group,” means selectively introducible and removable groups which protect carboxy groups against undesirable side reactions during synthetic procedures and includes all conventional carboxy protecting groups. Examples of carboxy protecting groups include methyl, ethyl, n-propyl, isopropyl, 1,1-dimethylpropyl, n-butyl, tert-butyl, phenyl, naphthyl, benzyl, diphenylmethyl, triphenylmethyl(trityl), para-nitrobenzyl, para-methoxybenzyl, acetylmethyl, benzoylmethyl, para-nitrobenzoylmethyl, para-bromobenzoylmethyl, 2-tetrahydropyranyl 2-tetrahydrofuranyl, 2,2,2-trichloroethyl cyclopropyl, cyclobutyl, cyclopentyl, cyclohexyl, methoxymethyl, methoxyethoxymethyl, arylalkoxyalkyl benzyloxymethyl 1,1-dimethyl-2-propenyl, 3-methyl-3-butenyl, allyl, and the like. Carboxy protecting groups can also be used as prodrug-forming groups.

The term “coupling catalyst” means palladium(0) complexes such as tetrakis(triphenylphosphine)palladium(0), tris(dibenzylideneacetone)dipalladium(0), allylpalladium chloride dimer, dipalladium tris(dibenzylidine acetone), and the like; palladium(II) salts such as palladium acetate, palladium chloride, and the like; palladium(II) complexes such as dichlorobis(triphenylphosphine)palladium(II), (1,1′-bis(diphenylphosphino)ferrocene)dichloropalladium(II), bis(acetato)bis(triphenylphosphine)palladium(II), bis(acetonitrile)dichloropalladium(II), and the like; nickel(0) complexes such as tetrakis(triphenylphosphine)nickel(0) and the like; and nickel(II) complexes such as dichlorobis(triphenylphosphine)nickel(II) and the like.

The term “cyano,” means —CN.

The term “cyanoalkyl,” means an alkyl group to which is attached at least one cyano substituent.

The term “cycloalkyl,” means a monovalent saturated cyclic or bicyclic hydrocarbon of three to fifteen carbons.

The term “cycloalkylalkyl,” means an alkyl group to which is attached at least one cycloalkyl group.

The term “halo,” means F (fluoride), Cl (chloride), Br (bromide), and I (iodide).

The term “haloalkyl,” means means an alkyl group to which is attached at least one halo substituent.

The term “heteroaryl,” means cyclic, aromatic five- and six-membered groups, wherein at least one atom is selected from the group consisting of nitrogen, oxygen, and sulfur, and the remaining atoms are carbon. The five-membered rings have two double bonds, and the six-membered rings have three double bonds. Heteroaryls are exemplified by furanyl, thienyl, pyrrolyl, oxazolyl, thiazolyl, imidazolyl, isoxazolyl, isothiazolyl, oxadiazolyl, oxadiazolyl, triazolyl, tetrazolyl, thiadiazolyl, pyridyl, pyridazinyl, pyrimidinyl, pyrazinyl, pyrazolyl, pyrrolyl, triazinyl, and the like. The heteroaryl groups of the invention are connected through a substitutable carbon or nitrogen (for imidazolyl or pyrrolyl) in the ring. The heteroaryl groups of the invention can be fused to an aryl group, a heterocyclyl, or another heteroaryl. Fused heteroaryls are exemplified by quinolinyl, isoquinolinyl, benzofuranyl, benzothienyl, indolyl, imidazo(2,1-b) (1,3)thiazolyl, and the like. The heteroaryl groups of the invention can be optionally substituted with 1-4 substituents independently selected from alkyl, alkenyl, alkynyl, alkoxyalkoxy, amino, aminoalkyl, alkylsulfanyl, alkylsulfonyl, cyano, cyanoalkyl, halo, haloalkyl, nitro, perfluoroalkyl, perfluoroalkoxy, oxo, —(CH₂)ₐC(O)R⁵, —(CH₂)ₐC(O)OR⁵, —(CH₂)ₐN(R⁵)C(O)R⁵, —(CH₂)ₐC(O)N(R⁵)₂, —(CH₂)ₐN(R⁵)C(O)N(R⁵)₂, —(CH₂)ₐOR⁵, —(CH₂)ₐSO₂R⁵, —(CH₂)ₐSR⁶, and —(CH₂)ₐR⁷.

The term “heteroarylalkyl,” means an alkyl group to which is attached at least one heteroaryl group.

The term “heteroarylene,” means a diradical formed by the removal of two hydrogen atoms from a heteroaryl, as defined directly above. Heteroarylenes are exemplified by isoxazol-3,4-diyl, isoxazol-3,5-diyl, isothiazol-3,4-diyl, isothiazol-3,5-diyl, oxazol-2,4-diyl, oxazol-2,5-diyl, oxazol-4,5-diyl, and the like.

The term “heterocyclyl,” means cyclic or bicyclic, non-aromatic, four-, five-, six-, or seven-membered rings containing at least one atom selected from the group consisting of oxygen, nitrogen, and sulfur. The four-membered rings have zero double bonds, the five-membered rings have zero or one double bonds, the six- and seven-membered rings have zero, one, or two double bonds; and the bicyclic heterocyclyls have zero to two double bonds. Heterocyclyls of the invention are exemplified by dihydropyranyl, dihydropyridinyl, 1,3-dioxolanyl, 1,4-dioxanyl, morpholinyl, piperazinyl, piperidinyl, pyrrolidinyl, tetrahydropyridinyl, thiomorpholinyl, and the like. The heterocyclyl groups of the invention can be fused to an aryl group, a heteroaryl, or another heterocyclyl. Fused heterocyclyls are exemplified by 1,3-benzodioxole, 2,3-dihydro-1,4-benzodioxine, and the like. The heterocyclyl groups of the invention are connected through a substitutable carbon or nitrogen atom in the ring. The heterocyclyl groups of the invention can be optionally substituted with 1-5 substituents independently selected from alkyl, alkenyl, alkynyl, alkoxyalkoxy, amino, aminoalkyl, alkylsulfanyl, alkylsulfonyl, cyano, cyanoalkyl, halo, haloalkyl, nitro, perfluoroalkyl, perfluoroalkoxy, oxo, —(CH₂)ₐC(O)R⁵, —(CH₂)ₐC(O)OR⁵, —(CH₂)ₐN(R⁵)C(O)R⁵, —(CH₂)ₐC(O)N(R⁵)₂, —(CH₂)ₐN(R⁵)C(O)N(R⁵)₂, —(CH₂)ₐOR⁵, —(CH₂)ₐSO₂R⁵, —(CH₂)ₐSR⁶, and —(CH₂)ₐR⁷.

The term “heterocyclene,” means a diradical formed by the removal of two hydrogen atoms from a heterocyclyl, as defined directly above. Heterocyclenes are exemplified by pyrrolidin-2,4-diyl, 1,3-dioxolan-2,4-diyl, and the like.

The term “hydroxy protecting group,” means selectively introducible and removable groups which protect hydroxy groups against undesirable side reactions during synthetic procedures. Examples of hydroxy protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, trimethylsilyl (TMS), triethylsilyl, 2-(trimethylsilyl)-ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, tert-butyl, 2,2,2-trichloroethyl, 2-trimethylsilylethyl, 1,1-dimethyl-2-propenyl, 3-methyl-3-butenyl, allyl, benzyl, para-methoxybenzyldiphenylmethyl, triphenylmethyl (trityl), tetrahydrofuryl methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, and the like. Hydroxy protecting group can also be used as prodrug-forming groups. Preferred hydroxy protecting groups for the practice of the invention are acetyl and benzoyl.

The term “nitro,” means —NO₂.

The term “oxo,” means a group formed by the replacement of two hydrogen atoms on the same carbon atom with a single oxygen atom.

The term “perfluoroalkyl,” means an alkyl group in which all of the hydrogen atoms have been replaced by fluoride atoms.

It is intended that the definition of any substituent or variable at a particular part in a molecule be independent of its definition elsewhere in the molecule. Thus, for example, substituents such as —(CH₂)ₐC(O)R⁵ represent —CH₂C(O)H, and —CH₂C(O)CH₃; and substituents such as —(CH₂)ₐN(R⁵)C(O)N(R⁵)₂ represent CH₂CH₂N(H)C(O)N(CH₃)(C₃H₇) and —CH₂N(CH₃)C(O)NH(CH₃), and the like.

The compounds of the invention can exist as therapeutically acceptable salts. The term “therapeutically acceptable salt,” means salts or zwitterionic forms of the compounds of the invention which are water or oil-soluble or dispersible, which are suitable for treatment of diseases without undue toxicity, irritation, and allergic response, which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, palmoate, pectinate, persulfate, 3-phenylpropionate, picrate, pivalate, propionate, succinate, tartrate, trichloroacetic, trifluoroacetic, phosphate, glutamate, bicarbonate, para-toluenesulfonate, and undecanoate. Also, amino groups in the compounds of the invention can be quaternized with as methyl, ethyl, propyl, and butyl chlorides, bromides and iodides; dimethyl, diethyl, dibutyl, and diamyl sulfates; decyl, lauryl, myristyl, and stearyl chlorides, bromides, and iodides; benzyl and phenethyl bromides. Examples of acids which can be employed to form therapeutically acceptable acid addition salts include inorganic acids such as hydrochloric, hydrobromic, sulphuric, and phosphoric and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary or tertiary amine. Therapeutically acceptable salts cations based on lithium, sodium, potassium, calcium, magnesium, and aluminum and nontoxic quaternary ammonia and amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of this invention can exist as therapeutically acceptable prodrugs. The term “therapeutically acceptable prodrug,” as used herein, represents those prodrugs of the compounds of this invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of this invention.

The term “prodrug,” as used herein, represents compounds, which are rapidly transformed in vivo to the parent compound of the above formula, for example, by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987.

Examples of compounds encompassed by Formula I include

a) a compound of formula (I) wherein D¹ is C≡C, Y¹ is isoxazol-3,5-diyl, A¹ is pyrid-2-yl, X is fluoride, and R¹ is hydrogen;
b) a compound of formula (I) wherein D¹ is C≡C, Y¹ is isoxazol-3,5-diyl, A¹ is quinol-3-yl, X is hydrogen, and R¹ is hydrogen;
c) a compound of formula (I) wherein D¹ is C≡C, Y¹ is isoxazol-3,5-diyl,
A¹ is quinol-2-yl, X is hydrogen, and R¹ is hydrogen;
d) a compound of formula (I) wherein D¹ is C≡C, Y¹ is isoxazol-3,5-diyl,
A¹ is quinol-4-yl, X is hydrogen, and R¹ is hydrogen;
e) a compound of formula (I) wherein D¹ is C≡C, Y¹ is isoxazol-3,5-diyl,
A¹ is 4-fluorophenyl, X is hydrogen, and R¹ is hydrogen;
f) a compound of formula (I) wherein D¹ is C≡C, Y¹ is isoxazol-3,5-diyl,
A¹ is pyrid-4-yl, X is hydrogen, and R¹ is hydrogen;
g) a compound of formula (I) wherein D¹ is C≡C, Y¹ is isoxazol-3,5-diyl,
A¹ is 4-cyanophenyl, X is hydrogen, and R¹ is hydrogen;
h) a compound of formula (I) wherein D¹ is C≡C, Y¹ is isoxazol-3,5-diyl,
A¹ is pyrid-3-yl, X is hydrogen, and R¹ is hydrogen;
i) a compound of formula (I) wherein D¹ is C≡C, Y¹ is isoxazol-3,5-diyl,
A¹ is thien-2-yl, X is hydrogen, and R¹ is hydrogen;
j) a compound of formula (I) wherein D¹ is C≡C, Y¹ is isoxazol-3,5-diyl,
A¹ is thiazol-2-yl, X is hydrogen, and R¹ is hydrogen;
k) a compound of formula (I) wherein D¹ is C≡C, Y¹ is isoxazol-3,5-diyl,
A¹ is 3,4-difluorophenyl, X is hydrogen, and R¹ is hydrogen;
l) a compound of formula (I) wherein D¹ is C≡C, Y¹ is isoxazol-3,5-diyl,
A¹ is 3-(trifluoromethyl)phenyl, X is hydrogen, and R¹ is hydrogen;
m) a compound of formula (I) wherein D¹ is C≡C, Y¹ is isoxazol-3,5-diyl,
A¹ is 3,4-dichlorophenyl, X is hydrogen, and R¹ is hydrogen;
n) a compound of formula (I) wherein D¹ is C≡C, Y¹ is isoxazol-3,5-diyl,
A¹ is 3-cyanophenyl, X is hydrogen, and R¹ is hydrogen;
o) a compound of formula (I) wherein D¹ is C≡C, Y¹ is isoxazol-3,5-diyl,
A¹ is 4-cyano-3-(methylsulfanyl)pyrid-2-yl, X is hydrogen, and R¹ is hydrogen;
p) a compound of formula (I) wherein D¹ is C≡C, Y¹ is isoxazol-3,5-diyl,
A¹ is thiazol-5-yl, X is hydrogen, and R¹ is hydrogen;
q) a compound of formula (I) wherein D¹ is C≡C, Y¹ is isoxazol-3,5-diyl,
A¹ is 6-chloroimidazo(2,1-b)thiazol-5-yl, X is fluoride, and R¹ is hydrogen;
r) a compound of formula (I) wherein D¹ is C≡C, Y¹ is isoxazol-3,5-diyl,
A¹ is thiazol-5-yl, X is fluoride, and R¹ is hydrogen;
s) a compound of formula (I) wherein D¹ is C≡C, Y¹ is isoxazol-3,5-diyl,
A¹ is thiazol-2-yl, X is fluoride, and R¹ is hydrogen;
t) a compound of formula (I) wherein D¹ is C≡C, Y¹ is isoxazol-3,5-diyl,
A¹ is 3,4-dichlorophenyl, X is fluoride, and R¹ is hydrogen;
u) a compound of formula (I) wherein D¹ is C≡C, Y¹ is isoxazol-3,5-diyl,
A¹ is pyrimidin-5-yl, X is fluoride, and R¹ is hydrogen;
v) a compound of formula (I) wherein D¹ is C≡C, Y¹ is isoxazol-3,5-diyl,
A¹ is 2-methyl-2H-tetrazol-5-yl, X is hydrogen, and R¹ is hydrogen;
w) a compound of formula (I) wherein D¹ is C≡C, Y¹ is isoxazol-3,5-diyl,
A¹ is 2-methyl-2H-tetrazol-5-yl; X is fluoride; and R¹ is hydrogen;
x) a compound of formula (I) wherein D¹ is C≡C, Y¹ is isoxazol-3,5-diyl,
A¹ is 2-chloroquinol-3-yl, X is hydrogen, and R¹ is hydrogen;
y) a compound of formula (I) wherein D¹ is C≡C, Y¹ is isoxazol-3,5-diyl,
A¹ is 3-methylbenzo(b)thien-2-yl, X is hydrogen, and R¹ is hydrogen;
z) a compound of formula (I) wherein D¹ is C≡C, Y¹ is isoxazol-3,5-diyl,
A¹ is pyrid-2-yl, X is hydrogen, and R¹ is hydrogen;
aa) a compound of formula (I) wherein D¹ is C≡C, Y¹ is isoxazol-3,5-diyl,
A¹ is quinol-3-yl, X is fluoride, and R¹ is hydrogen.

One particular embodiment of this invention is a subgenus of formula I which may be represented by the following formula: 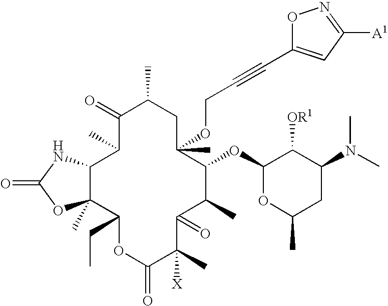
in which X is hydrogen or fluoride; R¹ is hydrogen, and A¹ is represented by aryl or heteroaryl. More specifically A¹ is phenyl, substituted phenyl, pyridyl, pyrimidinyl, thienyl, thiazolyl, quinolyl, benzothienyl, or imidazo (2,1-b) thiazolyl, in which any of said heterocycles may be further substituted. Even more particularly, A¹ is pyrid-2-yl, pyrid-3-yl, pyrid-4-yl, pyrimidin-2-yl, pyrimidin-4-yl or pyrimidin-5-yl.

In accordance with pharmaceutical compositions, methods of treatment, use as medicaments and as medicaments, the compounds can be administered alone to achieve an antibacterial effect or in combination with other antibacterial agents. The therapeutically effective dose level depends on factors such as the disorder being treated and the severity of the disorder; the activity of the compound used; the composition employed; the age, body weight, general health, sex, and diet of the patient; the time of administration; the route of administration; the rate of excretion of the compound; the duration of treatment; and drugs used in combination with or coincidentally with the compounds. The compounds can be administered orally, parenterally, nasally, rectally, vaginally, or topically in unit dosage formulations containing therapeutically acceptable excipients such as carriers, adjuvants, diluents, vehicles, or combinations thereof. The term “parenteral” includes infusion, subcutaneous, intravenous, intramuscular, and intrastemal injection.

The antibacterial effect of parenterally administered compounds can be controlled by slowing their absorption, such as, for example, by administration of injectable suspensions of crystalline, amorphous, or otherwise water-insoluble forms of the compounds; administration of the compounds as oleaginous solutions or suspensions; or administration of microencapsulated matrices of the compounds trapped within liposomes, microemulsions, or biodegradable polymers. In each case, the ratio of compound to excipient and the nature of the excipient influences the rate of release of the compound. Transdermal patches also provide controlled delivery of compounds using rate-controlling membranes. Conversely, absorption enhancers can be used to increase absorption of the compounds.

Solid dosage forms for oral administration of the compounds include capsules, tablets, pills, powders, and granules. These compositions can contain diluents, lubricants, and buffering agents. Tablets and pills can be prepared with release-controlling coatings, and sprays can optionally contain propellants. . Liquid dosage forms for oral administration of the compounds include emulsions, microemulsions, solutions, suspensions, syrups, and elixirs. These compositions can also contain adjuvants such as wetting, emulsifying, suspending, sweetening, flavoring, and perfuming agents.

Topical dosage forms of the compounds include ointments, pastes, creams, lotions, gels, powders, solutions, sprays, and inhalants. Suppositories for rectal or vaginal administration comprise compounds with a suitable nonirritating excipient. Ophthalmic formulations such as eye drops and eye are also contemplated as being within the scope of this invention.

The total daily dose of the compounds administered to a patient in single or divided doses can be in amounts from about 0.1 to about 200 mg/kg body weight or preferably from about 0.25 to about 100 mg/kg body weight. Single dose compositions contain these amounts or submultiples thereof to make up the daily dose.

### Determination of Biological Activity In Vitro Assay of Antibacterial Activity

Representative compounds were assayed in vitro for antibacterial activity as follows: twelve petri dishes containing successive aqueous dilutions of the test compound and 10 mL of sterilized Brain Heart Infusion (BHI) agar (Difco 0418-01-5) were prepared. Each plate was inoculated with 1:100 (or 1:10 for slow-growing Streptococcus strains) dilutions of the nine microorganisms shown in Table 1 using a Steers replicator block. The inoculated plates were incubated at about 35-37° C. for 20-24 hours. A control plate, using BHI agar containing no test compound, was also prepared and incubated at the beginning and end of each test. Finally, a plate containing Erythromycin A was prepared and incubated as another control and to provide test-to-test comparability.

After incubation, each plate was inspected. The minimum inhibitory concentration (MIC) was defined as the lowest concentration of drug yielding no growth, a slight haze, or sparsely isolated colonies on the inoculum spot as compared to the growth control. The compounds inhibited the growth of these bacteria with MIC's in a range of about 0.004 μg/mL to about 128 μg/mL; in a more preferred range, the compounds inhibited the growth of bacteria with MIC's in a range of about 0.004 μg/mL to about 2 μg/mL; and in a most preferred range, the compounds inhibited the growth of bacteria with MIC's in a range of about 0.004 μg/mL to about 4 μg/mL.

The results of this assay demonstrate the antibacterial activity of the compounds of the invention.

TABLE 1

Microorganism
Staphylococcus aureus ATCC 6538P
Staphylococcus aureus A5177
Streptococcus pyogenes EES61
Streptococcus pyogenes 930
Streptococcus pyogenes PIU 2548
Streptococcus pneumoniae ATCC 6303
Streptococcus pneumoniae 5737
Streptococcus pneumoniae 5649
Haemophilus influenzae DILL AMP R

### Gastrointestinal Tolerability Study

Example 3 and three reference compounds were investigated for their ability to produce nausea and emesis in conscious ferrets using the method as described in Drugs, 53(2), 206-234 (1997) and Cancer Treat. Rep., 66(1), 187-189 (1982). Each compound was administered to 6 ferrets by oral gavage at 30 mg/kg in 2 mL of ethanol and 4 mL of water. Following administration, the ferrets were observed for 90 minutes for signs of nausea and vomiting. Nausea was preceded by up to five of the following behaviors in the ferret: licking, down, flop, backing, and gag. From these behaviors a nausea score is determined for each compound, one point assigned for each behavior exhibited. The mean nausea score for a compound is the total number of nausea behaviors divided by the number of animals given the compound. Percent emesis is the total number of vomiting ferrets divided by the number of animals administered the compound. The results, shown in Table 2, demonstrate the unexpected gastrointestinal tolerance of Example 3.

TABLE 2

| | Dose | | | |
|---|---|---|---|---|
| Compound | (mg/kg) | No. Animals | % Emesis | Nausea Score |
| Erythromycin | 30 | 6 | 67% | 1.2 |
| Clarithromycin | 30 | 6 | 83% | 1.4 |
| Telithromycin | 30 | 6 | 50% | 1.8 |
| Example 3 | 30 | 6 | 0% | 0.17 |

This enhanced gastrointestinal tolerability represents a significant advantage for the compounds of this invention. These compounds will have an improved side effect profile when compared with the erythromycin derivatives of the prior art. Patients consuming these compounds will experience a reduced incidence of nausea, vomiting, gastrointestinal discomfort, cramping, and other GI side effects typically associated with erythromycin therapy. As used in this application, “enhanced gastrointestinal tolerance” refers to a reduced incidence of GI side effects in a patient population, and not to a total absence of GI side effects. As is well known to those skilled in the art, even placebo dosage forms made of sugar produce some measurable incidence of side effects. Thus an enhanced profile must be interpreted in light of the relevant art.

### Abbreviations

Abbreviations which have been used in the descriptions of the schemes and the examples that follow are: THF for tetrahydrofuran, DMF for N,N-dimethylformamide, DME for 1,2-dimethoxyethane, LDA for lithium diisopropylamide and DDQ for 2,3-dichloro-5,6-dicyanobenzoquinone.

### Synthetic Methods

The compounds can be prepared by employing reactions shown in Schemes 1-10. It will be readily apparent to one of ordinary skill in the art that the compounds can be synthesized by substitution of the appropriate reactants in these syntheses, and that the steps themselves can be conducted in varying order. It will also be apparent that protection and deprotection steps can be performed to successfully complete the syntheses of the compounds. A thorough discussion of protecting groups is provided in T. W. Greene and P. G. M. Wuts, Protective Groups in Organic Synthesis, 3rd edition, John Wiley & Sons, New York (1999). The groups X, A¹, D¹, Y¹, R¹, and R^{p} are defined hereinabove and the groups R, W¹, and X¹ are defined hereinbelow.

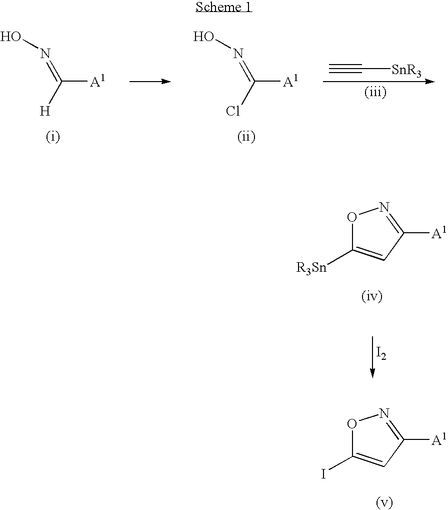

As shown in Scheme 1, conversion of compounds of formula (i) to compounds of formula (ii) can be achieved by treatment of the former with chlorinating agents. Examples of chlorinating agents include N-chlorosuccinimide and chlorine gas. Solvents useful for the reaction include DMF, THF, ethyl acetate, and mixtures thereof. The temperatures at which the reactions are conducted typically range from about 25° C. to about 40° C.

Conversion of compounds of formula (i) to compounds of formula (iv) can be achieved by in situ treatment of compounds of formula (ii), prepared as described above, with compounds of formula (iii) (R is C₁-C₄ alkyl) and a base. Examples of bases include sodium bicarbonate, sodium carbonate, triethylamine, and N,N-diisopropylethylamine. Solvents useful for the reaction include DMF, THF, ethyl acetate, and mixtures thereof. The temperatures at which the reactions are conducted typically range from about 25° C. to about 40° C.; and reaction times are typically from about 12 hours to about 48 hours.

Conversion of compounds of formula (iv) to compounds of formula (v) can be achieved by treatment of the former with iodine. Solvents useful for the reaction include THF, 1,4-dioxane, toluene, and mixtures thereof. The temperature at which the reactions are conducted is typically ambient; and reaction times are typically from about 2 hours to about 4 hours.

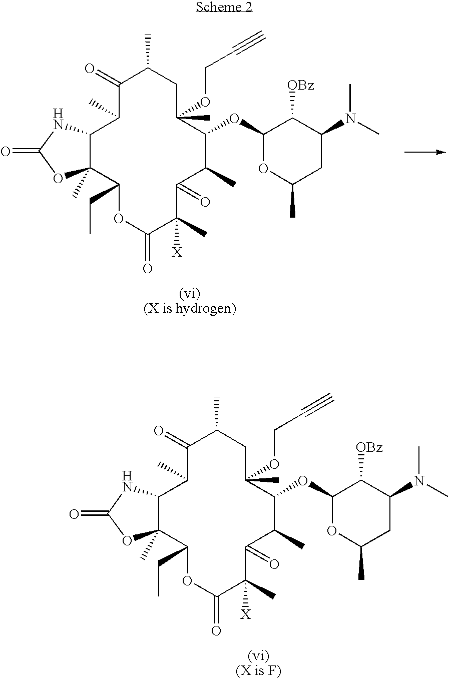

As shown in Scheme 2, compounds of formula (vi), wherein X is hydrogen, can be intraconverted to compounds of formula (vi), wherein X is fluoride, by treatment of the former with a fluorinating agent and, optionally, a base. Examples of fluorinating agents include 3,5-dichloro-1-fluoropyridinium tetrafluoroborate, N-fluorobenzenesulfonimide, 3,5-dichloro-1-fluoropyridinium triflate, and N-fluorobenzenesulfonimide, N-fluoro-N-methyl-para-toluenesulfonamide, N-fluoropyridinium triflate, or N-fluoroperfluoropiperidine and a base. Examples of bases include sodium hydride, potassium hydride, LDA, triethylamine, and N,N-diisopropylethylamine. Solvents useful for the reaction include THF, diethylether, and mixtures thereof. The temperatures at which the reactions are conducted typically range from about −78° C. to about 0° C.; and reaction times are typically from about 2 hours to about 24 hours.

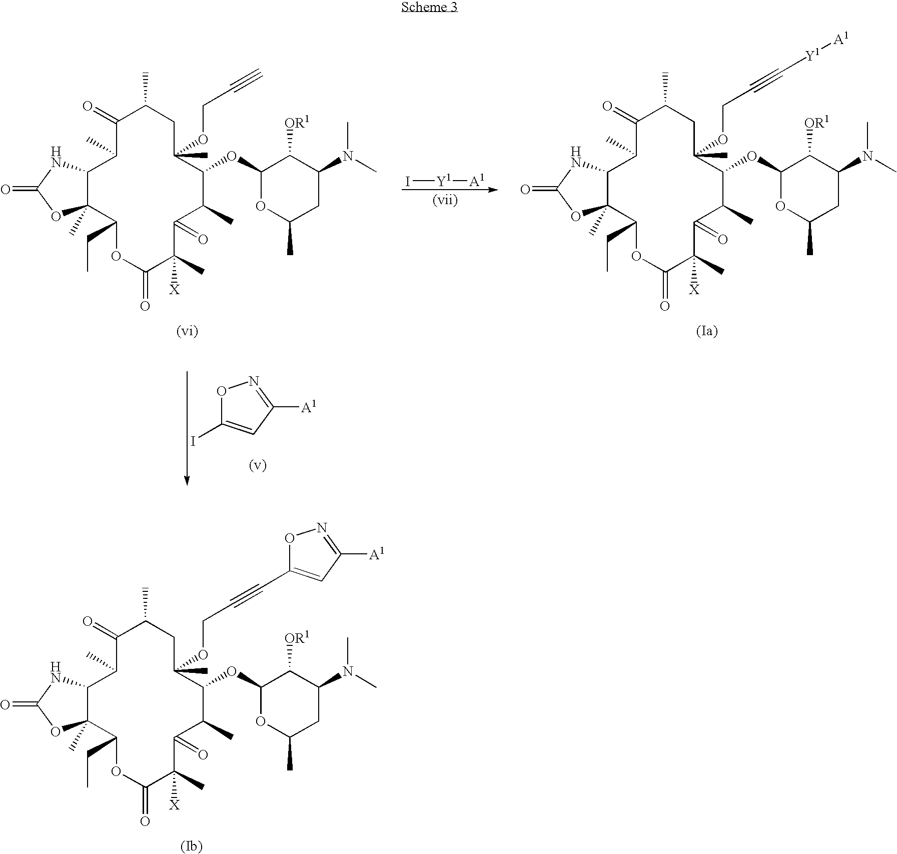

As shown in Scheme 3, compounds of formula (vi) can be converted to compounds of formula (Ia) by treatment of the former with compounds of formula (vii), a base, a coupling catalyst, and, optionally, an additive. Examples of bases include triethylamine and N,N-diisopropylethylamine. Examples of coupling catalysts include dichlorobis(triphenylphosphine)palladium(II), tris(dibenzylideneacetone)dipalladium(0), tetrakis(triphenylphosphine)palladium(0), and dichlorobis(triphenylphosphine)nickel(II). Examples of additives include triphenylphosphine, triphenylarsine, copper(I) iodide, and mixtures thereof. Solvents useful for the reaction include acetonitrile, THF, triethylamine, and mixtures thereof. The temperatures at which the reactions are conducted typically range from about 50° C. to about 80° C.; and reaction times are typically from about 12 hours to about 48 hours.

In a particular embodiment of this reaction, the compounds of formula (vi) can be treated with compounds of formula (v) to provide compounds of formula (Ib).

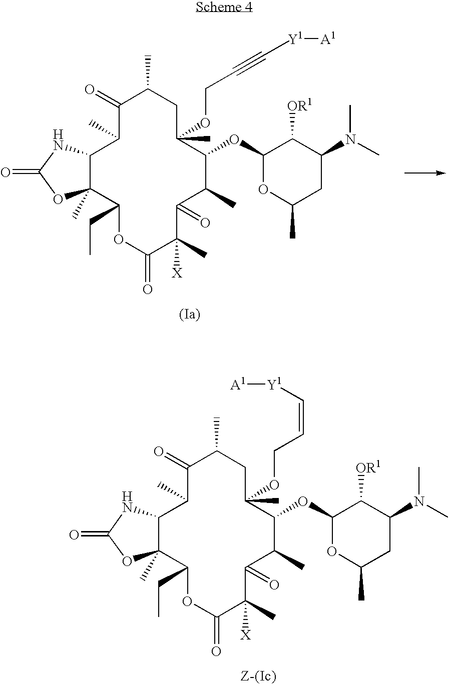

As shown in Scheme 4, compounds of formula (Ia), wherein D¹ is C≡C, can be converted to compounds of formula Z-(Ic), wherein D¹ is CH═CH in the Z configuration, by treatment of the former with hydrogen gas, a reduction catalyst, and, optionally, an additive. Examples of reduction catalysts include Lindlar catalyst and palladium on barium sulfate. An example of an additive is quinoline. Solvents useful for the reaction include C₁-C₄ alcohols such as methanol, ethanol, propanol, butanol, iso-propanol, tert-butanol, and the like, acetonitrile, THF, ethyl acetate, and mixtures thereof. The temperature at which the reactions are conducted is typically ambient; and reaction times are typically from about 1 hour to about 6 hours.

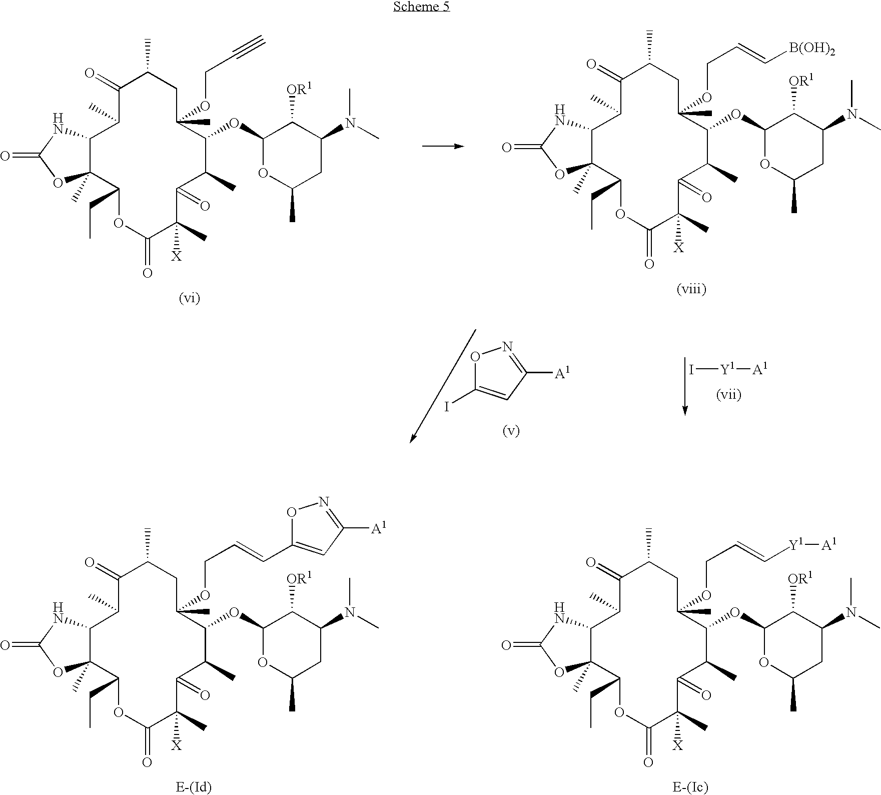

As shown in Scheme 5, compounds of formula (vi) can be converted to compounds of formula (viii) by treatment of the former with borane THF. Solvents useful for the reaction include THF, dioxane, diethylether, and mixtures thereof. The temperatures at which the reactions are conducted is typically about −20° C. to about 25° C.; and reaction times are typically from about 1 hour to about 6 hours.

Compounds of formula (viii) can be converted to compounds of formula E-(Ic), wherein D¹ is CH═CH in the E configuration, by treatment of the former with compounds of formula (vii), a coupling catalyst, a base, and, optionally, an additive. Examples of coupling catalysts include dichlorobis(triphenylphosphine)palladium(II), tris(dibenzylideneacetone)dipalladium(0), tetrakis(triphenylphosphine)palladium(0), and dichlorobis(triphenylphosphine)nickel(II). Examples of bases include sodium carbonate, potassium carbonate, cesium carbonate, triethylamine, and N,N-diisopropylethylamine. Examples of additives include triphenylphosphine, tributylphosphine, and triphenylarsine. Solvents useful for the reaction include acetonitrile, THF, DMF, DME, and mixtures thereof. The temperatures at which the reactions are conducted typically range from about 50° C. to about 80° C.; and reaction times are typically from about 12 hours to about 48 hours.

In a particular embodiment of this reaction, the compounds of formula (viii) can be treated with compounds of formula (v) to provide compounds of formula E-(Id).

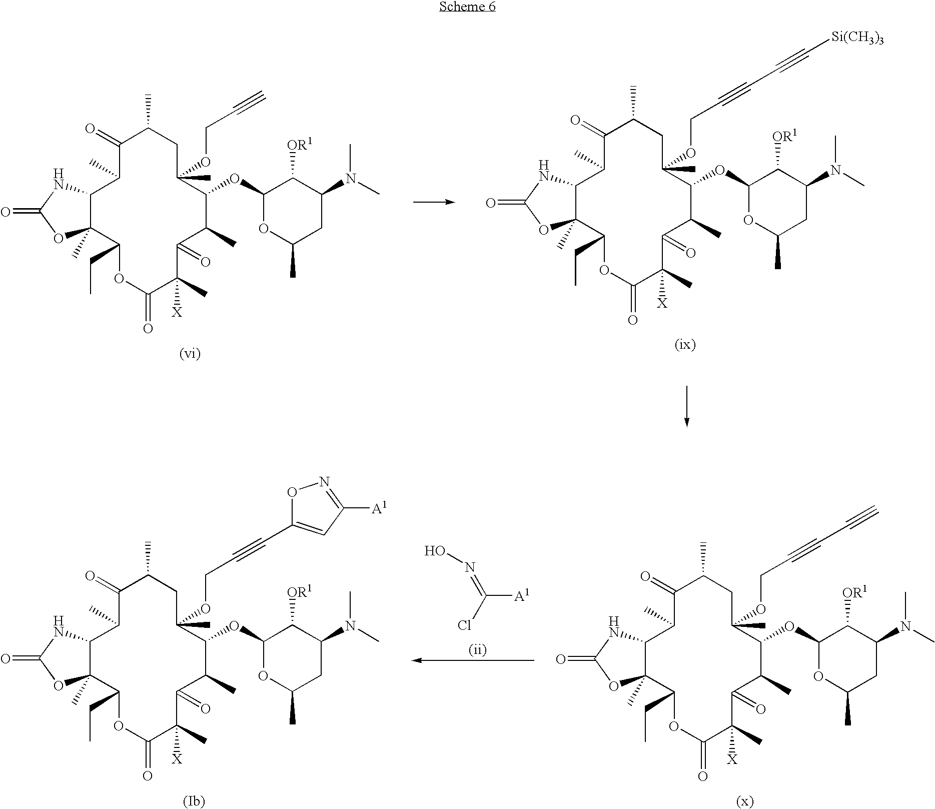

As shown in Scheme 6, conversion of compounds of formula (vi) to compounds of formula (ix) can be achieved by treatment of the former with 1-iodo-2-(trimethylsilyl)acetylene, a base, a coupling catalyst, and, optionally, an additive. Examples of bases include triethylamine and N,N-diisopropylethylamine. Examples of coupling catalysts include dichlorobis(triphenylphosphine)palladium(II), tris(dibenzylideneacetone)dipalladium(0), tetrakis(triphenylphosphine)palladium(0), and dichlorobis(triphenylphosphine)nickel(II). Examples of additives include copper(I) iodide, triphenylphosphine, and triphenylarsine. Solvents useful for the reaction include acetonitrile, triethylamine, THF, and mixtures thereof. The temperatures at which the reactions are conducted typically range from about 25° C. to about 80° C.; and reaction times are typically from about 6 hours to about 24 hours.

Conversion of compounds of formula (ix) to compounds of formula (x) can be achieved by treatment of the former with a base. Examples of bases include potassium carbonate and sodium carbonate. Solvents useful for the reaction include methanol or ethanol, and mixtures thereof. The temperature at which the reactions are conducted is typically ambient; and the reaction times are typically about 5-15 minutes.

Conversion of compounds of formula (x) to compounds of formula (Ib) can be achieved by treatment of the former with compounds of formula (ii) and a base. Examples of bases include sodium bicarbonate, sodium carbonate, triethylamine, and N,N-diisopropylethylamine. Solvents useful for the reaction include DMF, THF, ethyl acetate, and mixtures thereof. The temperatures at which the reactions are conducted typically range from about 25° C. to about 40° C.; and reaction times are typically from about 12 hours to about 48 hours.

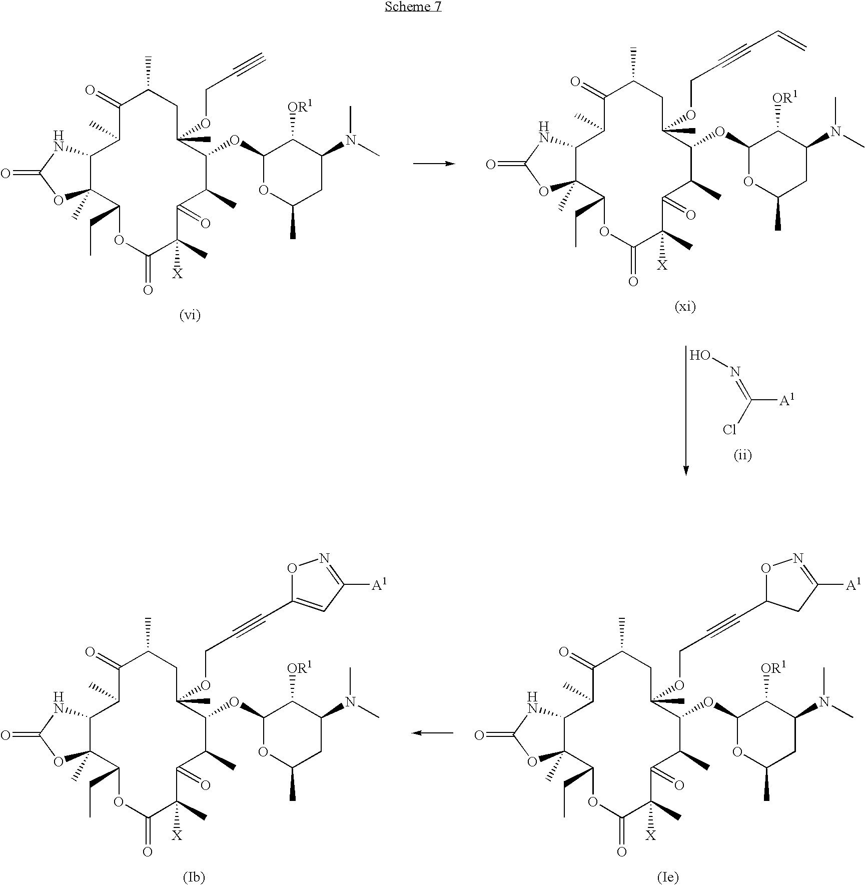

As shown in Scheme 7, conversion of compounds of formula (vi) to compounds of formula (xi) can be achieved by treatment of the former with vinyl bromide, a base, a coupling catalyst, and, optionally, an additive. Examples of bases include triethyl amine and N,N-diisopropylethylamine. Examples of coupling catalysts include dichlorobis(triphenylphosphine)palladium(II), tris(dibenzylideneacetone)dipalladium(0), tetrakis(triphenylphosphine)palladium(0), and dichlorobis(triphenylphosphine)nickel(II). Examples of additives include triphenylphosphine, triphenylarsine, and copper(I) iodide. Solvents useful for the reaction include acetonitrile, THF, triethylamine, and mixtures thereof. The temperatures at which the reactions are conducted typically range from about 25° C. to about 80° C.; and reaction times are typically from about 12 hours to about 48 hours.

Conversion of compounds of formula (xi) to compounds of formula (Ie) can be achieved by treatment of the former with compounds of formula (ii) and a base. Examples of bases include sodium bicarbonate, sodium carbonate, triethylamine, and N,N-diisopropylethylamine. Solvents useful for the reaction include DMF, THF, ethyl acetate, and mixtures thereof. The temperatures at which the reactions are conducted typically range from about 25° C. to about 40° C.; and reaction times are typically from about 12 hours to about 48 hours.

Conversion of compounds of formula (Ie) to compounds of formula (Ib) can be achieved by treatment of the former with oxidizing agents. Examples of oxidizing agents include manganese dioxide, barium manganate, and DDQ. Solvents useful for the reaction include THF, 1,4-dioxane, and mixtures thereof. The temperatures at which the reactions are conducted typically range from about 50° C. to about 100° C.; and reaction times are typically from about 12 hours to about 96 hours.

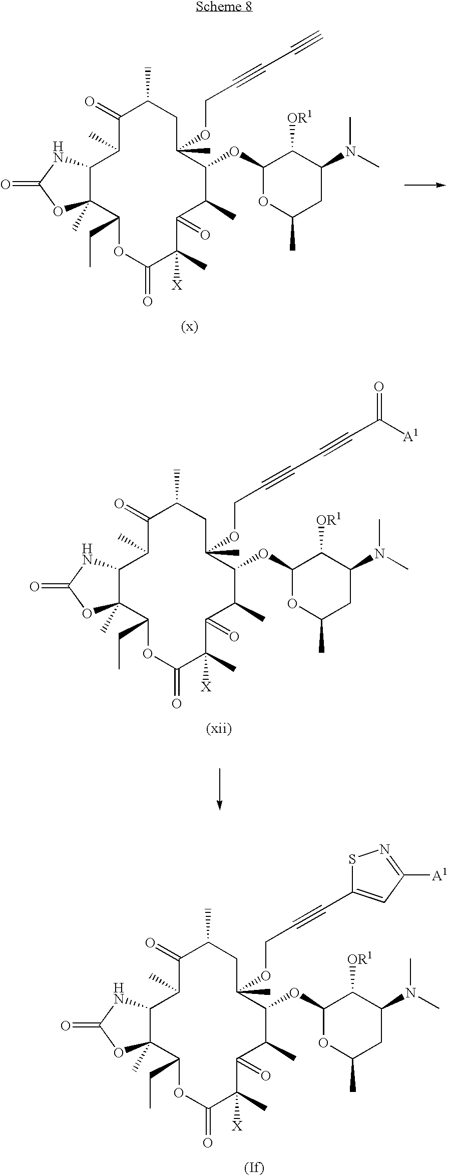

As shown in Scheme 8, compounds of formula (x) can be converted to compounds of formula (xii) by treatment of the former with comopunds of formula X¹—C(O)—A¹, wherein X¹ is Br or Cl, a coupling catalyst, a base, and, optionally, an additive. Examples of coupling catalysts include allylpalladium chloride dimer, tetrakis(triphenylphosphine)palladium(0), dichlorobis(triphenylphosphine)palladium(II), and dichlorobis(triphenylphosphine)nickel(II). Examples of bases include N,N,N,N-tetramethyl-1,8-naphthalenediamine (Proton-Sponge®), triethylamine and N,N-diisopropylethylamine. Examples of additives include triphenylphosphine, triphenylarsine, and copper(I) iodide. Solvents useful for the reaction include acetonitrile, THF, 1,4-dioxane, DME, triethylamine, and mixtures thereof. The temperatures at which the reactions are conducted typically range from about 25° C. to about 100° C.; and reaction times are typically from about 6 hours to about 24 hours.

Alternatively, compounds of formula (x) can be converted to compounds of formula (xii) by treatment of the former with compounds of formula W¹-A¹, wherein W¹ is halogen, —OSO₂CF₃, or —SnR³ (R is C₁-C₄ alkyl), carbon monoxide, a coupling catalyst, and, optionally, a base and an additive. Examples of coupling catalysts include allylpalladium chloride dimer, tetrakis(triphenylphosphine)palladium(0), dichlorobis(triphenylphosphine)palladium(II), and dichlorobis(triphenylphosphine)nickel(II). Examples of bases include N,N,N,N-tetramethyl-1,8-naphthalenediamine (Proton-Sponge®), triethylamine and N,N-diisopropylethylamine. Examples of additives include triphenylphosphine, triphenylarsine, and copper(I) iodide. Solvents useful for the reaction include acetonitrile, THF, 1,4-dioxane, DME, triethylamine, and mixtures thereof. The temperatures at which the reactions are conducted typically range from about 25° C. to about 100° C.; and reaction times are typically from about 6 hours to about 24 hours.

Conversion of compounds of formula (xii) to compounds of formula (If) can be achieved by treatment of the former with N-hydroxylamine-O-sulfonic acid, sodium hydrosulfide, and a base. Examples of bases include sodium bicarbonate, sodium carbonate, and potassium carbonate. Solvents useful for the reaction include C₁-C₄ alcohols, water, acetonitrile, THF, 1,4-dioxane, DME, and mixtures thereof. The temperatures at which the reactions are conducted typically range from about 0° C. to about 50° C.; and reaction times are typically from about 6 hours to about 24 hours.

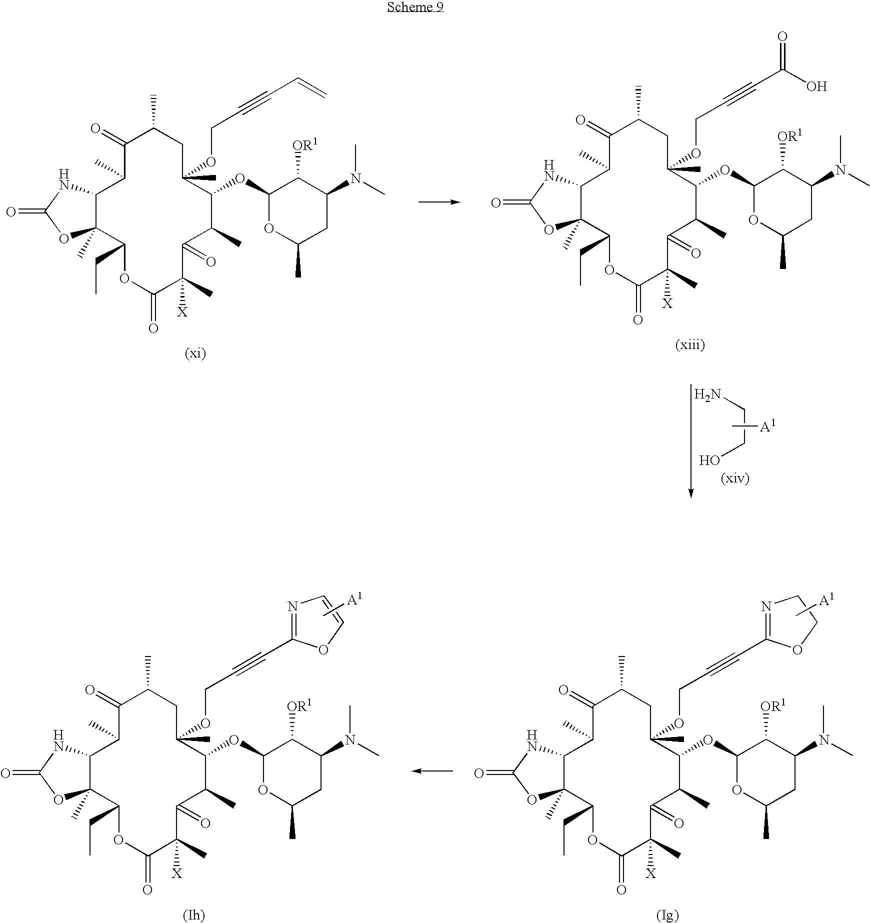

As Shown in Scheme 9, compounds of formula (xi) can be converted to compounds of formula (xiii) by treatment of the former with an oxidizing agent, and, optionally, an additive. Examples of oxidizing agents include potassium permanganate, sodium periodate, and ozone. Examples of additives include osmium tetroxide, N-methylmorpholine N-oxide, and hydrogen peroxide. Solvents useful for the reaction include acetonitrile, acetone, water, THF, and mixtures thereof. The temperatures at which the reactions are conducted typically range from about 0° C. to about 50° C.; and reaction times are typically from about 1 hour to about 4 hours.

Conversion of compounds of formula (xiii) to compounds of formula (Ig) can be achieved by treatment of the former with compounds of formula (xiv), triphenylphosphine, and an additive. Examples of additives include carbon tetrachloride, carbon tetrabromide, and diethyl azodicarboxylate. Solvents useful for the reaction include acetonitrile, THF, 1,4-dioxane, and mixtures thereof. The temperatures at which the reactions are conducted typically range from about 0° C. to about 50° C.; and reaction times are typically from about 1 hour to about 24 hours.

Conversion of compounds of formula (Ig) to compounds of formula (Ih) can be achieved by treatment of the former with oxidizing agents. Examples of oxidizing agents include manganese dioxide, barium manganate, and DDQ. Solvents useful for the reaction include THF, 1,4-dioxane, and mixtures thereof. The temperatures at which the reactions are conducted typically range from about 50° C. to about 100° C.; and reaction times are typically from about 12 hours to about 96 hours.

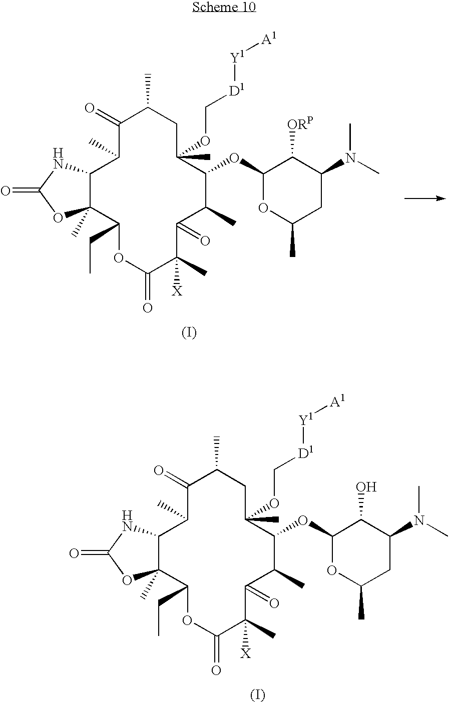

As shown in Scheme 10, compounds of formula (I), wherein R¹ is R^{p}, can be intraconverted to compounds of formula (I), wherein R¹ is hydrogen, by treatment of the former with methanol. The temperatures at which the reactions are conducted typically range from about 25° C. to about 65° C.; and reaction times are typically from about 2 hours to about 60 hours.

The invention will now be described in connection with other particularly preferred embodiments of Schemes 1-10, which are not intended to limit its scope. On the contrary, the invention covers all alternatives, modifications, and equivalents which are included within the scope of the claims. Thus, the following examples will illustrate an especially preferred practice of the invention, it being understood that the examples are for the purposes of illustration of certain preferred embodiments and are presented to provide what is believed to be the most useful and readily understood description of its procedures and conceptual aspects.

### EXAMPLE 1A

### Compound of Formula (vi) in Scheme 2: X is Hydrogen; R¹ is C₆H₅C(O)

A solution of Example 246 of commonly owned U.S. Pat. No. 5,866,549 in dichloromethane can be treated with 90% technical grade benzoic anhydride and triethylamine over 10 minutes, stirred for 48 hours, treated with saturated NaHCO₃, and stirred for 30 minutes. The layers can be separated, and the organic layer can be washed with water and brine, dried (Na₂SO₄), filtered, and concentrated. The concentrate can be triturated with a warm mixture of hexane and ethyl acetate and dried in a vacuum oven at ambient temperature to provide the desired product.

### EXAMPLE 1B

### Compound of Formula (ix) in Scheme 6: X is Hydrogen; R¹ is C₆H₅C(O)

A solution of Example 1A (15 g, 0.02 mol) in acetonitrile (150 mL) and triethylamine (75 mL) at room temperature was treated with dichlorobis(triphenylphosphine)palladium(II) (0.994 g, 1.4 mmol), copper(I) iodide (0.115 g, 0.6 mmol), and 1-iodo-2-(trimethylsilyl)acetylene (5.9 mL, 0.0385 mol), stirred at room temperature for 14 hours, and concentrated. The concentrate was suspended in ether and filtered through diatomaceous earth (Celite®). The filtrate was washed with water and brine, dried (Na₂SO₄), filtered, and concentrated. The concentrate was purified by flash column chromatography on silica gel with 85:15 hexanes/acetone to provide the desired product.

### EXAMPLE 1C

### Compound of Formula (x) in Scheme 6: X is Hydrogen; R¹ is C₆H₅C(O)

A solution of Example 1B (7.07 g, 8.44 mmol) in methanol (80 mL) at room temperature was treated with potassium carbonate (0.514 g, 4.22 mmol), stirred for 10 minutes, treated with ethyl acetate, washed with water and brine, dried (Na₂SO₄), filtered, and concentrated. The concentrate was purified by flash column chromatography on silica gel with 80:20 hexanes/acetone to provide the desired product. MS (ESI(+)) m/z 765 (M+H)⁺.

### EXAMPLE 1D

### Compound of Formula (x) in Scheme 6: X is Hydrogen; R¹ is Hydrogen

A solution of Example 1C (3.5 g, 4.57 mmol) in methanol (40 mL) at room temperature was stirred for 60 hours and concentrated. The concentrate was purified by flash column chromatography on silica gel with 98:1.5:1 dichloromethane/methanol/concentrated ammonium hydroxide to provide the desired product.

### EXAMPLE 1E

### Compound of Formula (x) in Scheme 6: X is Hydrogen; R¹ is CH₃C(O)

A solution of Example 1D (1.4 g, 2.12 mmol) and triethylamine (428 mg, 4.24 mmol) in dichloromethane (10 ml) at room temperature was treated with acetic anhydride (432 mg, 4.24 mmol), stirred for 3 hours, treated with dichloromethane (65 mL), washed with 5% NaHCO₃, brine, dried (Na₂SO₄), filtered, and concentrated. The concentrate was purified by flash column chromatography on silica gel with 70:30 hexanes/acetone to provide the desired product.

### EXAMPLE 2A

### Compound of Formula (vi) from Scheme 2: X is Fluoride; R¹ is C₆H₅C(O)

The desired compound was prepared as described in Example 1 of WO 99/21871, and substituting the instant Example 1A for the compound of formula (I) wherein R^{p} is benzoyl, R¹ is methyl, and X is F.

### EXAMPLE 2B

### Compound of Formula (ix) in Scheme 6: X is Fluoride; R¹ is C₆H₅C(O)

The desired product was prepared by substituting Example 2A for Example 1A in Example 1B.

### EXAMPLE 2C

### Compound of Formula (x) in Scheme 6: X is fluoride; R¹ is C₆H₅C(O)

The desired product was prepared by substituting Example 2B for Example 1B in Example 1C.

### EXAMPLE 3

### Compound of Formula (I): D¹ is C≡C, Y¹ is isoxazol-3,5-diyl, A¹ is pyrid-2-yl, X is fluoride, R¹ is hydrogen

### EXAMPLE 3A

### 2-(5-(tributylstannyl)-3-isoxazolyl)pyridine or

### Compound of Formula (iv) in Scheme 1: A¹ is pyrid-2-yl; R is n-butyl

A solution of 2-pyridinecarbaldehyde oxime (5.81 g, 47.6 mmol), tributyl(ethynyl)stannane (10.0 g, 31.7 mmol), and sodium bicarbonate (9.6 g, 114 mmol) in ethyl acetate (65 mL) and water (5 mL) was treated with N-chlorosuccinimide (6.36 g, 47.6 mmol), stirred for 18 hours at room temperature, treated with ethyl acetate, washed with 5% NaHCO₃, water, and brine, dried (MgSO₄), filtered, and concentrated. The concentrate was purified by flash column chromatography on silica gel with 95:5 hexanes/ethyl acetate to provide the desired product. MS m/z 435 (M+H)⁺.

### EXAMPLE 3B

### 2-(5-iodo-3-isoxazolyl)pyridine or

### Compound of Formula (v) in Scheme 1: A¹ is pyrid-2-yl

A solution of Example 3A (11.8 g, 27.3 mmol) in THF (200 mL) at room temperature was treated with iodine (7 g, 27.6 mmol), stirred for 2 hours, treated with diethyl ether, washed with saturated NaHCO₃ and saturated Na₂S₂O₃, dried (MgSO₄), filtered, and concentrated. The concentrate was purified by flash column chromatography on silica gel with 10:1 to 5:1 hexanes/ethyl acetate to provide the desired product. MS m/z 272 (M+H)⁺.

### EXAMPLE 3C

### Compound of Formula (Ib) in Scheme 3: A¹ is pyrid-2-yl; X is fluoride; R¹ is C₆H₅C(O)

A solution of Example 2A (1.715 g, 2.26 mmol) and Example 3B (737 mg, 2.71 mmol) in acetonitrile (10 mL) and triethylamine (2 mL) at room temperature was degassed, treated with dichlorobis(triphenylphosphine)palladium(II) (5 mole %), degassed again, stirred for 30 minutes, heated at 65° C. for 18 hours, concentrated to remove most of the solvent, treated with isopropyl acetate (500 mL), washed with saturated NaHCO₃, water, and brine, dried (Na₂SO₄), filtered, and concentrated. The concentrate was purified by flash column chromatography on silica gel with 2:1 hexanes/acetone to provide the desired product. MS m/z 903 (M+H)⁺.

### EXAMPLE 3D

### Compound of Formula (I): D¹ is C≡C, Y¹ is isoxazol-3,5-diyl, A¹ is pyrid-2-yl, X is fluoride, R¹ is hydrogen;

A solution of Example 3C (42.3 g, 46.8 mmol) in methanol (400 mL) was heated at reflux for 6 hours and concentrated. The concentrate was recrystallized from hexanes/acetone to provide the desired product. MS m/z 799 (M+H)⁺; ¹³C NMR (75 MHz, CDCl₃) δ 216.5, d (204.2 and 203.8), d (166.2 and 165.9), 163.3, 157.3, 153.7, 149.7, 148.7, 136.8, 124.5, 121.8, 107.1, 104.2, d (99.0 and 96.3), 96.1, 83.4, 80.7, 80.1, 78.7, 70.3, 69.8, 65.8, 58.1, 50.8, 44.0, 40.5, 40.2, 38.3, 37.5, 28.1, d (25.4 and 25.1), 22.2, 21.1, 20.3, 17.6, 15.3, 13.7, 13.2, 10.5; HRMS m/z calcd (M+H)⁺ for C₄₁H₅₅FN₄O₁₁: 799.3924. Found: 799.3924.

### EXAMPLE 4

### Compound of Formula (I): D¹ is C≡C, Y¹ is isoxazol-3,5-diyl, A¹ is quinol-3-yl, X is hydrogen, R¹ is hydrogen;

### EXAMPLE 4A

### 3-quinolinecarbaldehyde oxime or

### Compound of Formula (i) in Scheme 1: A¹ is quinol-3-yl

The desired product was prepared by substituting 3-quinolinecarbaldehyde for 4-formylbenzonitrile in Example 9A.

### EXAMPLE 4B

### 3-(5-(tributylstannyl)-3-isoxazolyl)quinoline or

### Compound of Formula (iv) in Scheme 1: A¹ is quinol-3-yl; R is n-butyl

The desired product was prepared by substituting Example 4A for Example 11A in Example 11B and purified by flash column chromatography on silica gel with 95:5 hexanes/acetone.

### EXAMPLE 4C

### 3-(5-iodo-3-isoxazolyl)quinoline or

### Compound of Formula (v) in Scheme 1: A¹ is quinol-3-yl

The desired product was prepared by substituting Example 4B for Example 11B in Example 11C.

### EXAMPLE 4D

### Compound of Formula (I): D¹ is C≡C, Y¹ is isoxazol-3,5-diyl, A¹ is quinol-3-yl; X is hydrogen; R¹ is C₆H₅C(O)

The desired product was prepared by substituting Example 4C for Example 11C in Example 11D and purified by flash column chromatography on silica gel with 90:10 hexanes/acetone.

### EXAMPLE 4E

### Compound of Formula (I): D¹ is C≡C, Y¹ is isoxazol-3,5-diyl, A¹ is quinol-3-yl, X is hydrogen, R¹ is hydrogen

A solution of Example 4D (198 mg, 0.212 mmol) in methanol (10 mL) was heated at 55° C. for 16 hours and concentrated. The concentrate was purified by flash column chromatography on silica gel with 98:1:1 dichloromethane/methanol/concentrated ammonium hydroxide to provide the desired product. ¹³C NMR (CDCl₃) δ 217.0 (C-9), 205.2 (C-3), 169.6 (C-1), 160.3, 157.9, 154.1, 148.8, 148.4, 134.2, 130.5, 129.5, 128.4, 127.5, 127.3, 121.8, 106.3, 103.1, 96.1, 83.6, 80.1, 77.4, 77.3, 72.4, 70.2, 69.5, 66.1, 58.1, 51.3, 51.1, 47.0, 44.7, 40.2, 38.6, 37.4, 28.5, 22.4, 21.1, 19.9, 18.0, 15.0, 14.5, 13.6, 13.6, 10.6; HRMS m/z calcd (M+H)⁺ calcd for C₄₅H₅₉N₄O₁₁: 831.4175. Found 831.4173.

### EXAMPLE 5

### Compound of Formula (I): D¹ is C≡C, Y¹ is isoxazol-3,5-diyl, Al is quinol-2-yl; X is hydrogen; R¹ is hydrogen

### EXAMPLE 5A

### 2-quinolinecarbaldehyde oxime or

### Compound of Formula (i) in Scheme 1: A¹ is quinol-2-yl

The desired product was prepared by substituting 2-quinolinecarbaldehyde for 4-formylbenzonitrile in Example 9A.

### EXAMPLE 5B

### 2-(5-(tributylstannyl)-3-isoxazolyl)quinoline or

### Compound of Formula (iv) in Scheme 1: A¹ is quinol-2-yl; R is n-butyl

The desired product was prepared by substituting Example 5A for Example 11A in Example 11B and purified by flash column chromatography on silica gel with 95:5 hexanes/acetone.

### EXAMPLE 5C

### 2-(5-iodo-3-isoxazolyl)quinoline or

### Compound of Formula (v) in Scheme 1: A¹ is quinol-2-yl

The desired product was prepared by substituting Example 5B for Example 11B in Example 11C and purified by flash column chromatography on silica gel with 95:5 hexanes/acetone.

### EXAMPLE 5D

### Compound of Formula (I): D¹ is C≡C, Y¹ is isoxazol-3,5-diyl, A¹ is quinol-2-yl; X is hydrogen; R¹ is C₆H₅C(O)

The desired product was prepared by substituting Example 5C for Example 11C in Example 11D and purified by flash column chromatography on silica gel with 75:25 hexanes/acetone.

### EXAMPLE 5E

### Compound of Formula (I): D¹ is C≡C, Y¹ is isoxazol-3,5-diyl, A¹ is quinol-2-yl; X is hydrogen; R¹ is hydrogen

The desired product was prepared by substituting Example 5D for Example 4D in Example 4E. ¹³C NMR (CDCl₃) δ 216.8 (C-9), 205.1 (C-3), 169.6 (C-1), 163.8, 157.8, 154.8, 148.2, 148.0, 136.8, 129.9, 129.8, 128.4, 127.6, 127.3, 119.2, 107.6, 103.3, 95.8, 83.5, 80.1, 77.5, 77.3, 72.7, 70.3, 69.7, 65.9, 58.1, 51.3, 51.1, 46.9, 44.7, 40.2, 38.6, 37.4, 28.2, 22.4, 21.2, 19.8, 18.0, 15.0, 14.5, 13.6, 13.6, 10.6; HRMS m/z (M+H)⁺ calcd for C₄₅H₅₉N₄O₁₁: 831.4175. Found 831.4175.

### EXAMPLE 6

### Compound of Formula (I): D¹ is C≡C, Y¹ is isoxazol-3,5-diyl, A¹ is quinol-4-yl; X is hydrogen; R¹ is hydrogen

### EXAMPLE 6A

### 4-quinolinecarbaldehyde oxime or

### Compound of Formula (i) in Scheme 1: A¹ is quinol-4-yl

The desired product was prepared by substituting 4-quinolinecarbaldehyde for 4-formylbenzonitrile in Example 9A.

### EXAMPLE 6B

### 4-(5-(tributylstannyl)-3-isoxazolyl)quinoline or

### Compound of Formula (iv) in Scheme 1: A¹ is quinol-4-yl; R is n-butyl

The desired product was prepared by substituting Example 6A for Example 11A in Example 11B and purified by flash column chromatography on silica gel with 95:5 hexanes/acetone.

### EXAMPLE 6C

### 4-(5-iodo-3-isoxazolyl)quinoline or

### Compound of Formula (v) in Scheme 1: A¹ is quinol-4-yl

The desired product was prepared by substituting Example 6B for Example 11B in Example 11C.

### EXAMPLE 6D

### Compound of Formula (I): D¹ is C≡C, Y¹ is isoxazol-3,5-diyl, A¹ is quinol-4-yl; X is hydrogen; R¹ is C₆H₅C(O)

The desired product was prepared by substituting Example 6C for Example 11C in Example 11D and purified by flash column chromatography on silica gel with 70:30 hexanes/acetone.

### EXAMPLE 6E

### Compound of Formula (I): D¹ is C≡C, Y¹ is isoxazol-3,5-diyl, A¹ is quinol-4-yl; X is hydrogen; R¹ is hydrogen

The desired product was prepared by substituting Example 6D for Example 4D in Example 4E. ¹³C NMR (CDCl₃) δ 217.0 (C-9), 205.2 (C-3), 169.6 (C-1), 160.8, 157.8, 153.8, 150.0, 148.8, 134.5, 130.0, 129.8, 127.7, 125.8, 125.4, 121.4, 109.1, 103.3, 96.4, 83.6, 80.1, 77.4, 77.3, 72.2, 70.2, 69.7, 65.8, 58.1, 51.3, 51.1, 47.0, 44.7, 40.2, 38.6, 37.4, 28.2, 22.4, 21.2, 19.9, 18.0, 15.0, 14.5, 13.6, 13.6, 10.5. HRMS m/z (M+H)⁺ calcd for C₄₅H₅₉N₄O₁₁: 831.4175. Found 831.4174.

### EXAMPLE 7

### Compound of Formula (I): D¹ is C≡C, Y¹ is isoxazol-3,5-diyl, A¹ is 4-fluorophenyl; X is hydrogen; R¹ is hydrogen

### EXAMPLE 7A

### 4-fluoro-N-hydroxybenzenecarboximidoyl chloride or

### Compound of Formula (ii) in Scheme 1: A¹ is 4-fluorophenyl

A solution of 4-fluorobenzaldoxime (1.0 g, 7.19 mmol) in DMF (6 mL) at room temperature was treated with HCl gas, collected from the head space of a bottle of 12M HCl, (5 mL) and N-chlorosuccinimide (0.960 g, 7.19 mmol) such that the reaction temperature was below 35° C., cooled to room temperature, treated with ethyl acetate, washed with water and brine, dried (Na₂SO₄), filtered, and concentrated to provide the desired product.

### EXAMPLE 7B

### Compound of Formula (I): D¹ is C≡C, Y¹ is isoxazol-3,5-diyl, A¹ is 4-fluorophenyl; X is hydrogen; R¹ is C₆H₅C(O)

A solution of Example 1C (0.12 g, 0.157 mmol) in ethyl acetate (1 mL) and water (one drop) was treated with Example 7A (37 mg, 0.212 mmol) and sodium bicarbonate (26.3 mg, 0.314 mmol), stirred at room temperature for 16 hours, and concentrated. The concentrate was purified by flash column chromatography on silica gel with 80:20 hexanes/acetone to provide the desired product.

### EXAMPLE 7C

### Compound of Formula (I): D¹ is C≡C, Y¹ is isoxazol-3,5-diyl, A¹ is 4-fluorophenyl; X is hydrogen; R¹ is hydrogen

The desired product was prepared by substituting Example 7B Example 11D in Example 11E and purified by flash column chromatography on silica gel with 50:50 hexanes/acetone. MS (ESI(+)) m/z 798 (M+H)⁺.

### EXAMPLE 8

### Compound of Formula (I): D¹ is C≡C, Y¹ is isoxazol-3,5-diyl, A¹ is pyrid-4-yl; X is hydrogen; R¹ is hydrogen

### EXAMPLE 8A

### 4-(5-(tributylstannyl)-3-isoxazolyl)pyridine or

### Compound of Formula (iv) in Scheme 1: A¹ is pyrid-4-yl; R is n-butyl

The desired product was prepared by substituting 4-pyridinecarbaldehyde oxime for Example 11A in Example 11B and purified by flash column chromatography on silica gel with 95:5 hexanes/acetone.

### EXAMPLE 8B

### 4-(5-iodo-3-isoxazolyl)pyridine or

### Compound of Formula (v) in Scheme 1: A¹ is pyrid-4-yl

The desired product was prepared by substituting Example 8A for Example 11B in Example 11C.

### EXAMPLE 8C

### Compound of Formula (I): D¹ is C≡C, Y¹ is isoxazol-3,5-diyl, A¹ is pyrid-4-yl; X is hydrogen; R¹ is C₆H₅C(O)

The desired product was prepared by substituting Example 8B for Example 11C in Example 11D.

### EXAMPLE 8D

### Compound of Formula (I): D¹ is C≡C, Y¹ is isoxazol-3,5-diyl, A¹ is pyrid-4-yl; X is hydrogen; R¹ is hydrogen

The desired product was prepared by substituting Example 8C for Example 4D in Example 4E. ¹³C NMR (CDCl₃) δ 217.0 (C-9), 205.1 (C-3), 169.5 (C-1), 2-160.7, 157.8, 2-155.5, 150.6, 136.0, 121.0, 106.3, 103.2, 96.3, 83.6, 80.1, 77.3, 77.2, 72.2, 70.2, 69.7, 65.9, 58.0, 51.2, 51.1, 47.0, 44.7, 40.2, 38.5, 37.4, 28.3, 22.4, 21.2, 19.9, 18.0, 15.1, 14.4, 13.6, 13.6, 10.6; HRMS m/z (M+H)⁺ calcd for C₄₁H₅₇N₄O₁₁: 781.4018. Found 781.4019.

### EXAMPLE 9

### Compound of Formula (I): D¹ is C≡C, Y¹ is isoxazol-3,5-diyl, A¹ is 4-cyanophenyl; X is hydrogen; R¹ is hydrogen

### EXAMPLE 9A

### 4-((hydroxyimino)methyl)benzonitrile or

### Compound of Formula (i) in Scheme 1: A¹ is 4-cyanophenyl

A solution of 4-formylbenzonitrile (2 g, 15.27 mmol) in methanol (6 mL) was treated with hydroxylamine hydrochloride (1.09 g, 15.72 mmol), stirred at room temperature for 24 hours, and concentrated. The concentrate was treated with 5% Na₂CO₃ and extracted with ethyl acetate. The extract was washed with brine, dried (Na₂SO₄), filtered, and concentrated to provide the desired oxime.

### EXAMPLE 9B

### 4-cyano-N-hydroxybenzenecarboximidoyl chloride or

### Compound of Formula (ii) in Scheme 1: A¹ is 4-cyanophenyl

A solution of Example 9A (0.3 g, 2.05 mmol) in DMF (1.5 mL) at room temperature was treated with HCl gas, collected from the head space of a bottle of 12M HCl, (5 mL) and N-chlorosuccinimide (0.273 g, 2.05 mmol) such that the reaction temperature was below 30° C., cooled to room temperature, treated with ice/water (10 mL), and filtered. The solid was washed with water and dried to provide the desired product as a white solid.

### EXAMPLE 9C

### Compound of Formula (I): D¹ is C≡C, Y¹ is isoxazol-3,5-diyl, A¹ is 4-cyanophenyl; X is hydrogen; R¹ is hydrogen

A solution of Example 1 D (0.1 g, 0.131 mmol) in benzene (1.5 mL) was treated with Example 9B (23.5 mg, 0.131 mmol) and triethylamine (19.8 mg, 0.196 mmol), stirred at room temperature for 18 hours, treated with additional Example 9B (19 mg, 0.105 mmol) and triethylamine (13.2 mg, 0.131 mmol), treated with ethyl acetate, washed with water and brine, dried (Na₂SO₄), filtered, and concentrated. The concentrate was purified by flash column chromatography on silica gel with 98:1:1 dichloromethane/methanol/concentrated ammonium hydroxide to provide the desired product. MS (ESI(+)) 805 (M+H)⁺.

### EXAMPLE 10

### Compound of Formula (I): D¹ is C≡C, Y¹ is isoxazol-3,5-diyl, A¹ is pyrid-3-yl; X is hydrogen; R¹ is hydrogen

### EXAMPLE 10A

### N-hydroxy-3-pyridinecarboximidoyl chloride or

### Compound of Formula (ii) in Scheme 1: A¹ is pyrid-3-yl

The desired product was prepared by substituting nicotinaldehyde oxime for Example 9A in Example 9B.

### EXAMPLE 10B

### Compound of Formula (I): D¹ is C≡C, Y¹ is isoxazol-3,5-diyl, A¹ is pyrid-3-yl; X is hydrogen; R¹ is hydrogen

The desired product was prepared by substituting Example 10A for Example 9B in Example 9C. ¹³C NMR (CDCl₃) δ 217.0 (C-9), 205.1 (C-3), 169.6 (C-1), 160.1, 157.8, 154.1, 151.1, 148.0, 134.2, 124.8, 123.8, 106.1, 103.2, 96.1, 83.6, 80.1, 77.3, 77.3, 72.3, 70.2, 69.7, 65.9, 58.0, 51.3, 51.1, 47.0, 44.7, 40.2, 38.6, 37.4, 28.2, 22.4, 21.2, 19.9, 18.0, 15.1, 14.5, 13.6, 13.6, 10.6; HRMS m/z (M+H)⁺ calcd for C₄₁H₅₇N₄O₁₁: 781.4018. Found 781.4015.

### EXAMPLE 11

### Compound of Formula (I): D¹ is C≡C, Y¹ is isoxazol-3,5-diyl, A¹ is thien-2-yl; X is hydrogen; R¹ is hydrogen

### EXAMPLE 11A

### 2-thiophenecarbaldehyde oxime or

### Compound of Formula (i) in Scheme 1: A¹ is thien-2-yl

The desired product was prepared by substituting 2-thiophenecarbaldehyde for 4-formylbenzonitrile in Example 9A.

### EXAMPLE 11B

### 3-(2-thienyl)-5-(tributylstannyl)isoxazole or

### Compound of Formula (iv) in Scheme 1: A¹ is thien-2-yl; R is n-butyl

A solution of Example 11A (3 g, 23.6 mmol) in ethyl acetate (70 mL) and water (100 μL) was treated with tributyl(ethynyl)stannane (6.82 mL, 23.6 mmol), N-chlorosuccinimide (3.13 g, 23.6 mmol) and sodium bicarbonate (4.75 g, 56.6 mmol), stirred at room temperature for 48 hours, treated with more ethyl acetate (100 mL), washed with water and brine, dried (Na₂SO₄), filtered, and concentrated. The concentrate was purified by flash column chromatography on silica gel with 98:2 hexanes/ethyl acetate to provide the desired product.

### EXAMPLE 11C

### 5-iodo-3-(2-thienyl)isoxazole or

### Compound of Formula (v) in Scheme 1: A¹ is thien-2-yl

A solution of Example 11B (1.05 g, 2.38 mmol) in THF (25 mL) at room temperature was treated with iodine (0.54 g, 2.14 mmol) in THF (15 mL) over 10 minutes, stirred for 3 hours, treated with ether (75 mL), washed with 5% NaHCO₃, 5% Na₂S₂O₃, and brine, dried (Na₂SO₄), filtered, and concentrated. The concentrate was treated with hexanes and filtered to provide the desired product.

### EXAMPLE 11D

### Compound of Formula (I): D¹ is C≡C, Y¹ is isoxazol-3,5-diyl, A¹ is thien-2-yl; X is hydrogen; R¹ is C₆H₅C(O)

A solution of Example 1A (503 mg, 0.68 mmol) in degassed acetonitrile (6 mL) and triethylamine (3 mL) was treated with Example 11C (216 mg, 0.782 mmol), dichlorobis(triphenylphosphine)palladium(II) (47.6 mg, 0.068 mmol) and copper(I) iodide (3.9 mg, 0.02 mmol), heated at 80° C. for 16 hours, and concentrated. The concentrate was purified by flash column chromatography on silica gel with 85:15 hexanes/acetone.

### EXAMPLE 11E

### Compound of Formula (I): D¹ is C≡C, Y¹ is isoxazol-3,5-diyl, A¹ is thien-2-yl; X is hydrogen; R¹ is hydrogen

A solution of Example 11D (370 mg, 0.416 mmol) in methanol (15 mL) was stirred at room temperature for 60 hours and concentrated. The concentrate was purified by flash column chromatography on silica gel with 98:1:1 dichloromethane/methanol/concentrated ammonium hydroxide to provide the desired product. ¹³C NMR (CDCl₃) δ 216.9 (C-9), 205.0 (C-3), 169.6 (C-1), 157.8, 157.8, 153.5, 130.2, 127.8, 127.7, 127.6, 106.4, 103.2, 95.7, 83.6, 80.1, 2-77.3, 72.4, 70.2, 69.7, 65.9, 58.0, 51.3, 51.1, 46.9, 44.7, 40.2, 38.6, 37.4, 28.2, 22.4, 21.2, 19.8, 18.0, 15.1, 14.5, 13.6, 13.6, 10.6; HRMS m/z (M+H)⁺ calcd for C₄₀H₅₆N₃O₁₁S: 786.3630. Found 786.3619.

### EXAMPLE 12

### Compound of Formula (I): D¹ is C≡C, Y¹ is isoxazol-3,5-diyl, A¹ is thiazol-2-yl; X is hydrogen; R¹ is hydrogen

### EXAMPLE 12A

### thiazole-2-carbaldehyde oxime or

### Compound of Formula (i) in Scheme 1: A¹ is thiazol-2-yl

The desired product was prepared by substituting thiazole-2-carbaldehyde for 4-formylbenzonitrile in Example 9A.

### EXAMPLE 12B

### 3-(thiazol-2-yl)-5-(tributylstannyl)isoxazole or

### Compound of Formula (iv) in Scheme 1: A¹ is thiazol-2-yl; R is n-butyl

The desired product was prepared by substituting Example 12A for Example 11A in Example 11B and purified by flash column chromatography on silica gel with 98:2 hexanes/ethyl acetate.

### EXAMPLE 12C

### 5-iodo-3-(thiazol-2-yl)isoxazole or

### Compound of Formula (v) in Scheme 1: A¹ is thiazol-2-yl

The desired product was prepared by substituting Example 12B Example 11B in Example 11C.

### EXAMPLE 12D

### Compound of Formula (I): D¹ is C≡C, Y¹ is isoxazol-3,5-diyl, A¹ is thiazol-2-yl; X is hydrogen; R¹ is C₆H₅C(O)

The desired product was prepared by substituting Example 12C for Example 11C in Example 11D and purified by flash column chromatography on silica gel with 80:20 hexanes/acetone.

### EXAMPLE 12E

### Compound of Formula (I): D¹ is C≡C, Y¹ is isoxazol-3,5-diyl, A¹ is thiazol-2-yl; X is hydrogen; R¹ is hydrogen

The desired product was prepared by substituting Example 12D for Example 4D in Example 4E. ¹³C NMR (CDCl₃) δ 216.9 (C-9), 205.0 (C-3), 169.7 (C-1), 158.6, 157.8, 155.0, 154.2, 143.8, 121.0, 106.5, 103.2, 96.4, 83.5, 80.1, 77.4, 77.3, 72.3, 70.2, 69.7, 65.9, 58.0, 51.3, 51.1, 46.9, 44.7, 40.2, 38.6, 37.4, 28.2, 22.3, 21.2, 19.8, 18.0, 15.0, 14.5, 13.6, 13.5, 10.5; HRMS m/z (M+H)⁺ calcd for C₃₉H₅₅N₄O₁₁S: 787.3583. Found 787.3581.

### EXAMPLE 13

### Compound of Formula (I): D¹ is C≡C, Y¹ is isoxazol-3,5-diyl, A¹ is 3,4-difluorophenyl; X is hydrogen; R¹ is hydrogen

### EXAMPLE 13A

### 3,4-difluorobenzaldehyde oxime or

### Compound of Formula (i) in Scheme 1: A¹ is 3,4-difluorophenyl

The desired product was prepared by substituting 3,4-difluorobenzaldehyde for 4-formylbenzonitrile in Example 9A.

### EXAMPLE 13B

### 3,4-difluoro-N-hydroxybenzenecarboximidoyl chloride or

### Compound of Formula (ii) in Scheme 1: A¹ is 3,4-difluorophenyl

The desired product was prepared by substituting Example 13A for Example 9A in Example 9B.

### EXAMPLE 13C

### Compound of Formula (I): D¹ is C≡C, Y¹ is isoxazol-3,5-diyl, A¹ is 3,4-difluorophenyl; X is hydrogen; R¹ is C₆H₅C(O)

The desired product was prepared by substituting Examples 1C and 13B for Examples 1D and 9B, respectively, in Example 9C and purified by flash column chromatography on silica gel with 85:15 hexanes/acetone.

### EXAMPLE 13D

### Compound of Formula (I): D¹ is C≡C, Y¹ is isoxazol-3,5-diyl, A¹ is 3,4-difluorophenyl; X is hydrogen; R¹ is hydrogen

The desired product was prepared by substituting Example 13C for Example 4D in Example 4E. ¹³C NMR (CDCl₃) δ 217.0 (C-9), 205.1 (C-3), 169.5 (C-1), 160.8, 157.8, 154.0, 152.7, 150.0 125.7, d (123.3 and 123.5), d (118.0 and 117.8), d (116.2 and 116.0), 106.2, 103.2, 95.9, 83.6, 80.1, 77.3, 77.3, 72.3, 70.2, 69.6, 66.0, 58.0, 51.3, 51.1, 47.0, 44.7, 40.2, 38.5, 37.4, 28.4, 22.4, 21.2, 19.9, 18.0, 15.1, 14.5, 13.6, 13.6, 10.6; HRMS m/z (M+H)⁺ calcd for C₄₂H₅₆F₂N₃O₁₁: 816.3877. Found 816.3886.

### EXAMPLE 14

### Compound of Formula (I): D¹ is C≡C, Y¹ is isoxazol-3,5-diyl, A¹ is 3-(trifluoromethyl)phenyl; X is hydrogen; R¹ is hydrogen

### EXAMPLE 14A

### N-hydroxy-3-(trifluoromethyl)benzenecarboximidoyl chloride or

### Compound of Formula (ii) in Scheme 1: A¹ is 3-(trifluoromethyl)phenyl

The desired product was prepared by substituting 3-(trifluoromethyl)benzaldehyde oxime for Example 9A in Example 9B.

### EXAMPLE 14B

### Compound of Formula (I): D¹ is C≡C, Y¹ is isoxazol-3,5-diyl, A¹ is 3-(trifluoromethyl)phenyl; X is hydrogen; R¹ is C₆H₅C(O)

The desired product was prepared by substituting Examples 1C and 14A for Examples 1D and 9B, respectively, in Example 9C and purified by flash column chromatography on silica gel with 85:15 hexanes/acetone.

### EXAMPLE 14C

### Compound of Formula (I): D¹ is C≡C, Y¹ is isoxazol-3,5-diyl, A¹ is 3-(trifluoromethyl)phenyl; X is hydrogen; R¹ is hydrogen

The desired product was prepared by substituting Example 14B for Example 4D in Example 4E and purified by flash column chromatography on silica gel with 98.5:1:0.5 dichloromethane/methanol/concentrated ammonium hydroxide. ¹³C NMR (CDCl₃) δ 216.9 (C-9), 205.1 (C-3), 169.5 (C-1), 161.5, 157.8, 154.1, 131.7, 130.1, 129.5, 126.7, d (126.6 and 128.6), 123.8, d (123.8 and 123.7), 106.3, 103.2, 96.0, 83.6, 80.1, 77.3, 72.3, 72.4, 70.2, 696, 66.0, 58.0, 51.3, 51.1, 47.0, 44.7, 40.2, 38.6, 37.4, 28.4, 22.4, 21.1, 20.0, 18.0, 15.1, 14.5, 13.6, 13.6, 10.6; HRMS m/z (M+H)⁺ calcd for C₄₃H₅₇F₃N₃O₁₁: 848.3940. Found 848.3948.

### EXAMPLE 15

### Compound of Formula (I): D¹ is C≡C, Y¹ is isoxazol-3,5-diyl, A¹ is 3,4-dichlorophenyl; X is hydrogen; R¹ is hydrogen

### EXAMPLE 15A

### 3,4-dichloro-N-hydroxybenzenecarboximidoyl chloride or

### Compound of Formula (ii) in Scheme 1: A¹ is 3,4-dichlorophenyl

The desired product was prepared by substituting 3,4-dichlorobenzaldehyde oxime for Example 9A in Example 9B.

### EXAMPLE 15B

### Compound of Formula (I): D¹ is C≡C, Y¹ is isoxazol-3,5-diyl, A¹ is 3,4-dichlorophenyl; X is hydrogen; R¹ is C₆H₅C(O)

The desired product was prepared by substituting Examples 1C and 15A for Examples 1D and 9B, respectively, in Example 9C and purified by flash column chromatography on silica gel with 85:15 hexanes/acetone.

### EXAMPLE 15C

### Compound of Formula (I): D¹ is C≡C, Y¹ is isoxazol-3,5-diyl, A¹ is 3,4-dichlorophenyl; X is hydrogen; R¹ is hydrogen

The desired product was prepared by substituting Example 15B for Example 14B in Example 14C. ¹³C NMR (CDCl₃) δ 217.0 (C-9), 205.1 (C-3), 169.5 (C-1), 160.7, 157.9, 154.1, 134.3, 133.3, 130.9, 128.7, 128.6, 126.1, 106.1, 103.3, 96.1, 83.6, 80.1, 77.3, 72.3, 70.2, 69.7, 65.9, 58.0, 51.2, 51.1, 47.0, 44.7, 40.2, 38.5, 37.4, 28.2, 22.4, 21.2, 19.9, 18.0, 15.1, 14.5, 13.6, 13.6, 10.6; HRMS m/z (M+H)⁺ calcd for C₄₂H₅₆C₁₂N₃O₁₁: 848.3286. Found 848.3303.

### EXAMPLE 16

### Compound of Formula (I): D¹ is C≡C, Y¹ is isoxazol-3,5-diyl, A¹ is 3-cyanophenyl; X is hydrogen; R¹ is hydrogen

### EXAMPLE 16A

### 3-((hydroxyimino)methyl)benzonitrile or

### Compound of Formula (i) in Scheme 1: A¹ is 3-cyanophenyl

The desired product was prepared by substituting 3-formylbenzonitrile for 4-formylbenzonitrile in Example 9A.

### EXAMPLE 16B

### 3-cyano-N-hydroxybenzenecarboximidoyl chloride or

### Compound of Formula (ii) in Scheme 1: A¹ is 3-cyanophenyl

The desired product was prepared by substituting Example 16A for Example 9A in Example 9B.

### EXAMPLE 16C

### Compound of Formula (I): D¹ is C≡C, Y¹ is isoxazol-3,5-diyl, A¹ is 3-cyanophenyl; X is hydrogen; R¹ is C₆H₅C(O)

The desired product was prepared by substituting Examples 1C and 16B for Examples 1D and 9B, respectively, in Example 9C and purified by flash column chromatography on silica gel with 85:15 hexanes/acetone.

### EXAMPLE 16D

### Compound of Formula (I): D¹ is C≡C, Y¹ is isoxazol-3,5-diyl, A¹ is 3-cyanophenyl; X is hydrogen; R¹ is hydrogen

The desired product was prepared by substituting Example 16C for Example 4D in Example 4E. ¹³C NMR (CDCl₃) δ 217.1 (C-9), 205.1 (C-3), 169.5 (C-1), 160.8, 157.8, 154.4, 133.3, 131.1, 130.3, 130.0, 129.9, 117.1, 113.3, 106.1, 103.3, 96.2, 83.6, 80.1, 77.3, 72.2, 70.2, 69.7, 65.9, 58.0, 51.2, 51.1, 47.1, 44.7, 40.2, 38.5, 37.4, 28.2, 22.4, 21.2, 19.9, 18.0, 15.2, 14.4, 13.6, 13.6, 10.6; HRMS m/z (M+H)⁺ calcd for C₄₃H₅₇N₄O₁₁: 805.4018. Found 805.4012.

### EXAMPLE 17

### Compound of Formula (I): D¹ is C≡C, Y¹ is isoxazol-3,5-diyl, A¹ is 4-cyano-3-(methylsulfanyl)pyrid-2-yl; X is hydrogen; R¹ is hydrogen

### EXAMPLE 17A

### 6-((hydroxyimino)methyl)-2-(methylsulfanyl)nicotinonitrile or

### Compound of Formula (i) in Scheme 1: A¹ is 4-cyano-3-(methylsulfanyl)pyrid-2-yl

The desired product was prepared by substituting 6-formyl-2-(methylsulfanyl)nicotinonitrile for 4-formylbenzonitrile in Example 9A.

### EXAMPLE 17B

### 5-cyano-N-hydroxy-6-(methylsulfanyl)-2-pyridinecarboximidoyl chloride or

### Compound of Formula (ii) in Scheme 1: A¹ is 4-cyano-3-(methylsulfanyl)pyrid-2-yl

The desired product was prepared by substituting Example 17A for Example 9A in Example 9B.

### EXAMPLE 17C

### Compound of Formula (I): D¹ is C≡C, Y¹ is isoxazol-3,5-diyl, A¹ is 4-cyano-3-(methylsulfanyl)pyrid-2-yl; X is hydrogen; R¹ is CH₃C(O)

A solution of Example 1E (125 mg, 0.179 mmol) in ethyl acetate (2 mL) at room temperature was treated with Example 17B (61 mg, 0.267mmol) and sodium bicarbonate (44.8 mg, 0.534 mmol), stirred for 2 hours, treated with additional Example 17B (202 mg, 0.89 mmol) and sodium bicarbonate (30.0 mg, 0.3576 mmol) added simultaneously in 4 portions over 7 hours, treated with ethyl acetate, washed with water and brine, dried (Na₂SO₄), filtered, and concentrated. The concentrate was purified by flash column chromatography on silica gel with 60:40 hexanes/acetone to provide the desired product.

### EXAMPLE 17D

### Compound of Formula (I): D¹ is C≡C, Y¹ is isoxazol-3,5-diyl, A¹ is 4-cyano-3-(methylsulfanyl)pyrid-2-yl; X is hydrogen; R¹ is hydrogen

A solution of Example 17C (130 mg, 0.145 mmol) in methanol (10 mL) was stirred at room temperature for 16 hours, concentrated, and dried to constant weight to provide the desired product. MS (ESI(+)) 852 (M+H)⁺.

### EXAMPLE 18

### Compound of Formula (I): D¹ is C≡C, Y¹ is isoxazol-3,5-diyl, A¹ is thiazol-5-yl; X is hydrogen; R¹ is hydrogen

### EXAMPLE 18A

### 2,4-dibromothiazole-5-carbaldehyde

A mixture of thiazolidine-2,4-dione (4.54 g, 34.88 mmol) and phosphorus oxybromide (50 g, 174.4 mmol) was treated with DMF (3.05 mL, 39.41 mmol), stirred at room temperature for 30 minutes, heated at 80° C. for 30 minutes, heated at 108° C. until hydrogen bromide evolution ceased (approximately 7 hours), cooled to room temperature, treated with ice/water (300 mL), and extracted with dichloromethane. The extract was washed with 5% aqueous NaHCO₃ and brine, dried (MgSO₄), filtered, and concentrated. The concentrate was purified by flash column chromatography on silica gel with 98:2 hexanes/ethyl acetate to provide the desired product.

### EXAMPLE 18B

### thiazole-5-carbaldehyde

The desired product was prepared from Example 18A as described in Syn. Comm., 25(24), 4081-4086 (1995).

### EXAMPLE 18C

### thiazole-5-carbaldehyde oxime or

### Compound of Formula (i) in Scheme 1: A¹ is thiazol-5-yl

The desired product was prepared by substituting Example 18B for 4-formylbenzonitrile in Example 9A.

### EXAMPLE 18D

### 3-(thiazol-5-yl)-5-(tributylstannyl)isoxazole or

### Compound of Formula (iv) in Scheme 1: A¹ is thiazol-5-yl; R is n-butyl

The desired product was prepared by substituting Example 18C for Example 11A in Example 11B.

### EXAMPLE 18E

### 5-iodo-3-(thiazol-5-yl)isoxazole or

### Compound of Formula (v) in Scheme 1: A¹ is thiazol-5-yl

The desired product was prepared by substituting Example 18D for Example 11B in Example 11C.

### EXAMPLE 18F

### Compound of Formula (I): D¹ is C≡C, Y¹ is isoxazol-3,5-diyl, A¹ is thiazol-5-yl; X is hydrogen; R¹ is C₆H₅C(O)

The desired product was prepared by substituting Example 18E for Example 11C in Example 11D and purified by flash column chromatography on silica gel with 80:20 hexanes/acetone.

### EXAMPLE 18G

### Compound of Formula (I): D¹ is C≡C, Y¹ is isoxazol-3,5-diyl, A¹ is thiazol-5-yl; X is hydrogen; R¹ is hydrogen

The desired product was prepared by substituting Example 18F for Example 4D in Example 4E. ¹³C NMR (CDCl₃) δ 217.1 (C-9),205.1 (C-3), 169.6 (C-1), 157.8, 155.4, 154.6, 154.1, 143.4 106.3, 103.2, 96.4, 83.6, 80.1, 77.3, 72.1, 70.2, 69.7, 65.9, 58.0, 53.4, 51.2, 51.1, 47.0, 44.7, 40.2, 38.6, 37.4, 28.2, 22.4, 21.1, 19.8, 18.0, 15.1, 14.5, 13.6, 13.6, 10.6; HRMS m/z (M+H)⁺ calcd for C₃₉H₅₅N₄O₁₁S: 787.3588. Found 787.3583.

### EXAMPLE 19

### Compound of Formula (I): D¹ is C≡C, Y¹ is isoxazol-3,5-diyl, A¹ is 6-chloroimidazo(2,1-b)thiazol-5-yl; X is fluoride; R¹ is hydrogen

### EXAMPLE 19A

### 6-chloroimidazo(2,1-b)thiazole-5-carbaldehyde oxime or

### Compound of Formula (i) in Scheme 1: A¹ is 6-chloroimidazo(2,1-b)thiazol-5-yl

The desired product was prepared by substituting 6-chloroimidazo(2,1-b)thiazole-5-carbaldehyde for 4-formylbenzonitrile in Example 9A.

### EXAMPLE 19B

### 6-chloro-5-(5-(tributylstannyl)-3-isoxazolyl)imidazo(2,1-b)thiazole or

### Compound of Formula (iv) in Scheme 1: A¹ is 6-chloroimidazo(2,1-b)thiazol-5-yl; R is n-butyl

The desired product was prepared by substituting Example 19A for Example 11A in Example 11B.

### EXAMPLE 19C

### 6-chloro-5-(5-iodo-3-isoxazolyl)imidazo(2,1-b)thiazole or

### Compound of Formula (v) in Scheme 1: A¹ is 6-chloroimidazo(2,1-b)thiazol-5-yl

The desired product was prepared by substituting Example 19B for Example 11B in Example 11C.

### EXAMPLE 19D

### Compound of Formula (I): D¹ is C≡C, Y¹ is isoxazol-3,5-diyl, A¹ is 6-chloroimidazo(2,1-b)thiazol-5-yl; X is fluoride; R¹ is C₆H₅C(O)

The desired product was prepared by substituting Examples 2A and 19C for Examples 1A and 11C, respectively, in Example 11D and purified by flash column chromatography on silica gel with 80:20 hexanes/acetone.

### EXAMPLE 19E

### Compound of Formula (I): D¹ is C≡C, Y¹ is isoxazol-3,5-diyl, A¹ is 6-chloroimidazo(2,1-b)thiazol-5-yl; X is fluoride; R¹ is hydrogen

The desired product was prepared by substituting Example 19D for Example 4D in Example 4E and purified by flash column chromatography on silica gel with 98:2 dichloromethane/methanol. ¹³C NMR (CDCl₃) δ 216.7 (C-9), d (204.3 and 204.0) (C-3), d (166.4 and 166.3) (C-1), 157.3, 153.4, 152.6, 149.5, 134.0, 121.9, 112.8, 105.5, 104.4, 99.0, 96.6, 96.3, 83.4, 80.7, 80.0, 78.8, 72.1, 70.3, 69.3, 66.8, 58.0, 50.7, 44.1, 40.6, 40.2, 38.3, 37.5, 28.1, 25.4, 25.1, 22.3, 21.2, 20.4, 17.7, 15.3, 13.8, 13.3, 10.7; HRMS m/z (M+H)⁺ calcd for C₄₁H₅₄ClFN₅O₁₁S: 878.3208. Found 878.3199.

### EXAMPLE 20

### Compound of Formula (I): D¹ is C≡C, Y¹ is isoxazol-3,5-diyl, A¹ is thiazol-5-yl; X is fluoride; R¹ is hydrogen

### EXAMPLE 20A

### Compound of Formula (I): D¹ is C≡C, Y¹ is isoxazol-3,5-diyl, A¹ is thiazol-5-yl; X is fluoride; R¹ is C₆H₅C(O)

The desired product was prepared by substituting Examples 2A and 18E for Examples 1A and 11C, respectively, in Example 11D and purified by flash column chromatography on silica gel with 80:20 hexanes/acetone.

### EXAMPLE 20B

### Compound of Formula (I): D¹ is C≡C, Y¹ is isoxazol-3,5-diyl, A¹ is thiazol-5-yl; X is fluoride; R¹ is hydrogen

The desired product was prepared by substituting Example 20A for Example 4D in Example 4E. ¹³C NMR (CDCl₃) δ 216.9 (C-9), d (204.3 and 203.9) (C-3), d (166.1 and 166.8) (C-1), 157.4, 155.5, 154.6, 154.0, 143.5, 125.8, 106.3, 104.2, d (99.0 and 96.2), 96.8, 83.5, 80.8, 80.0, 78.7, 72.0, 70.3, 69.8, 65.8, 58.0, 50.7, 44.1, 40.5, 40.2, 38.3, 37.5, 28.1, d (25.4 and 25.1), 22.2, 21.2, 20.3, 17.7, 15.4, 13.7, 13.3, 10.6; HRMS m/z (M+H)⁺ calcd for C₃₉H₅₄N₄O₁₁FS: 805.3494. Found 805.3466.

### EXAMPLE 21

### Compound of Formula (I): D¹ is C≡C, Y¹ is isoxazol-3,5-diyl, A¹ is thiazol-2-yl; X is fluoride; R¹ is hydrogen

### EXAMPLE 21A

### Compound of Formula (I): D¹ is C≡C, Y¹ is isoxazol-3,5-diyl, A¹ is thiazol-2-yl; X is fluoride; R¹ is C₆H₅C(O)

The desired product was prepared by substituting Examples 2A and 12C for Examples 1A and 11C, respectively, in Example 11D and purified by flash column chromatography on silica gel with 80:20 hexanes/acetone.

### EXAMPLE 21B

### Compound of Formula (I): D¹ is C≡C, Y¹ is isoxazol-3,5-diyl, A¹ is thiazol-2-yl; X is fluoride; R¹ is hydrogen

The desired product was prepared by substituting Example 21A for Example 14B in Example 14C. ¹³C NMR (CDCl₃) δ 216.6 (C-9), d (204.2 and 203.8) (C-3), d (166.1 and 165.9) (C-1), 158.6, 157.3, 155.9, 154.2, 143.8, 121.0, 106.5, 104.2, d (98.8 and 96.2), 96.8, 83.4, 80.8, 80.0, 78.7, 72.2, 70.3, 69.8, 65.8, 58.1, 50.8, 44.1, 40.5, 40.2, 38.3, 37.5, 28.1, d (25.3 and 25.1), 22.2, 21.2, 20.2, 17.6, 15.3, 13.7, 13.2, 10.5; HRMS m/z (M+H)⁺ calcd for C₃₉H₅₄FN₄O₁₁S: 805.3488. Found 805.3484.

### EXAMPLE 22

### Compound of Formula (I): D¹ is C≡C, Y¹ is isoxazol-3,5-diyl, A¹ is 3,4-dichlorophenyl; X is fluoride; R¹ is hydrogen

### EXAMPLE 22A

### Compound of Formula (I): D¹ is C≡C, Y¹ is isoxazol-3,5-diyl, A¹ is 3,4-dichlorophenyl; X is fluoride; R is C₆H₅C(O)

The desired product was prepared by substituting Examples 2C and 15A for Examples 1D and 9B, respectively, in Example 9C and purified by flash column chromatography on silica gel with 85:15 hexanes/acetone.

### EXAMPLE 22B

### Compound of Formula (I): D¹ is C≡C, Y¹ is isoxazol-3,5-diyl, A¹ is 3,4-dichlorophenyl; X is fluoride; R¹ is hydrogen

The desired product was prepared by substituting Example 22A for Example 4D in Example 4E. ¹³C NMR (CDCl₃) δ 216.8 (C-9), d (204.3 and 203.9) (C-3), d (166.0 and 166.8) (C-1), 160.7, 157.4, 154.1, 134.3, 133.3, 131.0, 128.7, 128.5, 126.1, 106.2, 104.2, d (98.8 and 96.3), 96.5, 83.5, 80.7, 80.0, 78.8, 72.2, 70.3, 69.8, 65.9, 58.1, 50.7, 44.1, 40.5, 40.2, 38.3, 37.4, 28.2, d (25.4 and 25.1), 22.3, 21.2, 20.4, 17.6, 15.4, 13.7, 13.2, 10.6; HRMS m/z (M+H)⁺ calcd for C₄₂H₅₅C₁₂FN₃O₁₁: 866.3192. Found 866.3196.

### EXAMPLE 23

### Compound of Formula (I): D¹ is C≡C, Y¹ is isoxazol-3,5-diyl, A¹ is pyrimidin-5-yl; X is fluoride; R¹ is hydrogen

### EXAMPLE 23A

### 5-pyrimidinecarbaldehyde

A solution of 5-bromopyrimidine (12 g, 0.075 mol) in THF (500 mL) at −100° C. was treated with 2.5M n-butyllithium in hexanes (30.2 mL, 79 mmol) over 35 minutes, stirred for 15 minutes at −100° C., treated with ethyl formate (6.7 mL, 0.0825 mol) over 15 minutes, stirred for 15 minutes at −95° C., treated with 1M HCl in ether (79 mL, 0.0787 mol) over 10 minutes, warmed to room temperature over 1 hour, and concentrated. The concentrate was treated with dichloromethane, and the resulting solution was washed with water and brine, dried (MgSO₄), filtered, and concentrated to provide the desired product.

### EXAMPLE 23B

### 5-pyrimidinecarbaldehyde oxime or

### Compound of Formula (i) in Scheme 1: A¹ is pyrimidin-5-yl

The desired product was prepared by substituting Example 23A for 4-formylbenzonitrile and substituting dichloromethane for ethyl acetate in the work-up to provide the desired product.

### EXAMPLE 23C

### N-hydroxy-5-pyrimidinecarboximidoyl chloride or

### Compound of Formula (ii) in Scheme 1: A¹ is pyrimidin-5-yl

The desired product was prepared by substituting Example 23B for Example 9A in Example 9B.

### EXAMPLE 23D

### Compound of Formula (I): D¹ is C≡C, Y¹ is isoxazol-3,5-diyl, A¹ is pyrimidin-5-yl; X is fluoride; R¹ is C₆H₅C(O)

The desired product was prepared by substituting Examples 2C and 23C for Examples 1D and 9B, respectively, in Example 9C and purified by flash column chromatography on silica gel with 80:20 hexanes/acetone.

### EXAMPLE 23E

### Compound of Formula (I): D¹ is C≡C, Y¹ is isoxazol-3,5-diyl, A¹ is pyrimidin-5-yl; X is fluoride; R¹ is hydrogen

The desired product was prepared by substituting Example 23D for Example 14B in Example 14C. ¹³C NMR (CDCl₃) δ 216.9 (C-9), d (204.3 and 204.0) (C-3), d (166.1 and 166.8) (C-1), 159.7, d (157.6 and 157.4), 154.8, 154.7, 154.6, 123.3, 105.8, 104.2, d (99.0 and 96.2), 97.2, 83.5, 80.8, 80.0, 78.7, 71.9, 70.2, 69.8, 65.9, 58.0, 50.7, 44.1, 40.5, 40.2, 38.3, 37.5, 28.2, d (25.4 and 25.1), 22.3, 21.2, 20.4, 17.6, 15.4, 13.7, 13.2, 10.6; HRMS n/z (M+H)⁺ calcd for C₄₀H₅₅FN₅O₁₁: 800.3879. Found 800.3876.

### EXAMPLE 24

### Compound of Formula (I): D¹ is C≡C, Y¹ is isoxazol-3,5-diyl, A¹ is 2-methyl-2H-tetrazol-5-yl; X is hydrogen; R¹ is hydrogen

### EXAMPLE 24A

### 2-methyl-2H-tetrazole

A solution of sodium hydroxide (14.2 g, 0.357 mol) in water (600 mL) at room temperature was treated with 1H-tetrazole (25 g, 0.357 mol) until homogeneous, treated with dichloromethane (600 mL), dimethyl sulfate (47.2 g, 0.375 mol), and N-tetrabutylammonium bromide (5.7 g, 0.0178 mol), and stirred for 14 hours. The organic layer was separated and concentrated, and the concentrate was distilled to provide the desired product (143° C./1 atm).

### EXAMPLE 24B

### 2-methyl-2H-tetrazole-5-carbaldehyde oxime or

### Compound of Formula (i) in Scheme 1: A¹ is 2-methyl-2H-tetrazol-5-yl

A solution of 2M lithium diisopropyl amide in heptane/THF/ethylbenzene (6.55 mL, 13.09 mmol) in THF (8.5 mL) at −75° C. was treated with Example 24A (1g, 11.9 mmol) in THF (10 mL) over 3 minutes, stirred for 30 minutes, treated with DMF (3.7 mL, 47.84 mmol) over 10 minutes, stirred for 5 minutes, warmed to room temperature, treated with hydroxylamine hydrochloride (1.15 g, 17.85 mmol) in methanol (6 mL) over 2 minutes, stirred for 20 hours, and concentrated. The concentrate was treated with ethyl acetate, washed with 5% NaHCO₃, water, and brine, dried (Na₂SO₄), filtered, and concentrated to provide the desired product.

### EXAMPLE 24C

### N-hydroxy-2-methyl-2H-tetrazole-5-carboximidoyl chloride or

### Compound of Formula (ii) in Scheme 1: A¹ is 2-methyl-2H-tetrazol-5-yl

The desired product was prepared by substituting Example 24B for Example 9A in Example 9B.

### EXAMPLE 24D

### Compound of Formula (I): D¹ is C≡C, Y¹ is isoxazol-3,5-diyl, A¹ is 2-methyl-2H-tetrazol-5-yl; X is hydrogen; R¹ is C₆H₅C(O)

The desired product was prepared by substituting Examples 1C and 24C for Examples 1D and 9B, respectively, in Example 9C and purified by flash column chromatography on silica gel with 80:20 hexanes/acetone.

### EXAMPLE 24E

### Compound of Formula (I): D¹ is C≡C, Y¹ is isoxazol-3,5-diyl, A¹ is 2-methyl-2H-tetrazol-5-yl; X is hydrogen; R¹ is hydrogen

The desired product was prepared by substituting the product from Example 24D for Example 14B in Example 14C. ¹³C NMR (CDCl₃) δ 217.1 (C-9), 205.0 (C-3), 169.7. (C-1), 157.8, 155.8, 154.6, 153.1, 107.4, 103.2, 96.8, 83.5, 80.2, 77.4, 77.3, 72.1, 70.2, 69.6, 65.9, 58.0, 51.3, 51.1, 46.9, 44.7, 40.2, 39.8, 38.6, 37.4, 28.2, 22.3, 21.1, 19.7, 17.9, 14.9, 14.5, 13.6, 13.5, 10.5; HRMS m/z (M+H)⁺ calcd for C₃₈H₅₆N₇O₁₁: 786.4038. Found 786.4016.

### EXAMPLE 25

### Compound of Formula (I): D¹ is C≡C, Y¹ is isoxazol-3,5-diyl, A¹ is 2-methyl-2H-tetrazol-5-yl; X is fluoride; R¹ is hydrogen

### EXAMPLE 25A

### Compound of Formula (I): D¹ is C≡C, Y¹ is isoxazol-3,5-diyl, A¹ is 2-methyl-2H-tetrazol-5-yl; X is fluoride; R¹ is C₆H₅C(O)

The desired product was prepared by substituting Example 2C for Example 1C in Example 24D.

### EXAMPLE 25B

### Compound of Formula (I): D¹ is C≡C, Y¹ is isoxazol-3,5-diyl, A¹ is 2-methyl-2H-tetrazol-5-yl; X is fluoride; R¹ is hydrogen

The desired product was prepared by substituting Example 25A for Example 4D in Example 4E. ¹³C NMR (CDCl₃) δ 217.1 (C-9), 205.0 (C-3), 169.7 (C-1), 157.8, 155.8, 154.6, 153.1, 107.4, 103.2, 96.8, 83.5, 80.2, 77.4, 77.3, 72.1, 70.2, 69.6, 65.9, 58.0, 51.3, 51.1, 46.9, 44.7, 40.2, 39.8, 38.6, 37.4, 28.2, 22.3, 21.1, 19.7, 17.9, 14.9, 14.5, 13.6, 13.5, 10.5; HRMS m/z (M+H)⁺ calcd for C₃₈H₅₅N₇O₁₁F: 804.3944. Found 804.3922.

### EXAMPLE 26

### Compound of Formula (I): D¹ is C≡C, Y¹ is isoxazol-3,5-diyl, A¹ is 2-chloroquinol-3-yl; X is hydrogen; R¹ is hydrogen

### EXAMPLE 26A

### 2-chloro-3-quinolinecarbaldehyde oxime or

### Compound of Formula (i) in Scheme 1: A¹ is 2-chloroquinol-3-yl

The desired product was prepared by substituting 2-chloro-3-quinolinecarbaldehyde for 4-formylbenzonitrile in Example 9A.

### EXAMPLE 26B

### 2-chloro-N-hydroxy-3-quinolinecarboximidoyl chloride or

### Compound of Formula (ii) in Scheme 1: A¹ is 2-chloroquinol-3-yl

The desired product was prepared by substituting Example 26A for Example 9A in Example 9B.

### EXAMPLE 26C

### Compound of Formula (I): D¹ is C≡C, Y¹ is isoxazol-3,5-diyl, A¹ is 2-chloroquinol-3-yl; X is hydrogen; R¹ is CH₃C(O)

A solution of Example 1E (125 mg, 0.1788 mmol) in benzene (1.5 ml) was treated with Example 26B (51mg, 0.213mmol) and triethylamine (30.5, 0.302 mmol), added in 3 portions over 30 minutes, stirred at room temperature for 20 hours and concentrated. The concentrate was purified by flash column chromatography on silica gel with 99:1 dichloromethane/methanol to provide the desired product.

### EXAMPLE 26D

### Compound of Formula (I): D¹ is C≡C, Y¹ is isoxazol-3,5-diyl, A¹ is 2-chloroquinol-3-yl; X is hydrogen; R¹ is hydrogen

The desired product was prepared by substituting Example 26C for Example 17C in Example 17D. ¹³C NMR (CDCl₃) δ 216.8 (C-9), d (204.2 and 203.8) (C-3), d (166.2 and 165.0) (C-1), 157.3, 156.8, 154.5, 153.1, 107.3 104.2, d (99.0 and 96.3), 97.2, 83.4, 80.8, 80.0, 78.6, 72.0, 70.3, 69.8, 65.8, 58.0, 50.8, 44.0, 40.6, 40.2, 39.8, 38.4, 37.5, 28.1, d (25.3 and 25.0), 22.2, 21.2, 20.2, 17.6, 15.3, 13.7, 13.2, 10.5; HRMS m/z (M+H)⁺ calcd for C₃₉H₅₃FN₄O₁₁S: 809.3973. Found 809.3966.

### EXAMPLE 27

### Compound of Formula (I): D¹ is C≡C, Y¹ is isoxazol-3,5-diyl, A¹ is 3-methylbenzo(b)thien-2-yl; X is hydrogen; R¹ is hydrogen

### EXAMPLE 27A

### 3-methylbenzo(b)thiophene-2-carbaldehyde oxime or

### Compound of Formula (i) in Scheme 1: A¹ is 3-methylbenzo(b)thien-2-yl

The desired product was prepared by substituting 3-methylbenzo(b)thiophene-2-carbaldehyde for 4-formylbenzonitrile in Example 9A.

### EXAMPLE 27B

### N-hydroxy-3-methylbenzo(b)thiophene-2-carboximidoyl chloride or

### Compound of Formula (ii) in Scheme 1: A¹ is 3-methylbenzo(b)thien-2-yl

The desired product was prepared by substituting Example 27A for Example 9A in Example 9B.

### EXAMPLE 27C

### Compound of Formula (I): D¹ is C≡C, Y¹ is isoxazol-3,5-diyl, A¹ is 3-methylbenzo(b)thien-2-yl; X is hydrogen; R¹ is C₆H₅C(O)

The desired product was prepared by substituting Examples 1C and 27B for Examples 1D and 9B, respectively, in Example 9C and purified by flash column chromatography on silica gel with 85:15 hexanes/acetone.

### EXAMPLE 27D

### Compound of Formula (I): D¹ is C≡C, Y¹ is isoxazol-3,5-diyl, A¹ is 3-methylbenzo(b)thien-2-yl; X is hydrogen; R¹ is hydrogen

The desired product was prepared by substituting Example 27C for Example 14B in Example 14C. ¹³C NMR (CDCl₃) δ 217.0 (C-9), 205.2 (C-3), 173.3, 169.6 (C-1), 158.1, 157.8, 153.5, 140.7, 139.4, 133.0, 125.5, 124.3, 122.6, 122.3, 108.2, 103.1, 96.0, 83.6, 80.1, 77.3, 77.2, 72.5, 70.2, 69.6, 65.9, 58.1, 51.8, 51.3, 51.0, 46.8, 44.7, 40.2, 38.6, 37.4, 28.4, 22.4, 21.2, 19.7, 18.0, 14.9, 14.5, d (13.6 and 13.6), 13.2, 10.5; HRMS m/z (M+H)⁺ calcd for C₄₅H₆₀N₃O₁₁S: 850.3943. Found 850.3947.

### EXAMPLE 28

### Compound of Formula (I): D¹ is C≡C, Y¹ is isoxazol-3,5-diyl, A¹ is pyrid-2-yl; X is hydrogen; R¹ is hydrogen

### EXAMPLE 28A

### Compound of Formula (I): D¹ is C≡C, Y¹ is isoxazol-3,5-diyl, A¹ is pyrid-2-yl; X is hydrogen; R¹ is C₆H₅C(O)

A solution of Example 1A (1.42 g, 2.0 mmol) and Example 3B (408 mg, 3 mmol) in acetonitrile (6 mL) and triethylamine (1 mL) at room temperature was degassed, treated with tris(dibenzylideneacetone)dipalladium(0) (91 mg, 0.1 mmol) and triphenylarsine (61 mg, 0.2 mmol), degassed again, heated at 80° C. for 24 hours, and concentrated. The concentrate was purified by flash column chromatography on silica gel with 2:1 hexanes/acetone to provide the desired product.

### EXAMPLE 28B

### Compound of Formula (I): D¹ is C≡C, Y¹ is isoxazol-3,5-diyl, A¹ is pyrid-2-yl; X is hydrogen; R¹ is hydrogen

A solution of Example 28A (1.24 g, 1.4 mmol) in methanol (20 mL) was heated at reflux for 6 hours and concentrated. The concentrate was purified by flash column chromatography on silica gel with 100:1:0.5 dichloromethane/methanol/concentrated ammonium hydroxide to provide the desired product. MS m/z 781 (M+H)⁺; ¹³C NMR (75 MHz, CDCl₃) δ 216.7, 205.0, 169.6, 163.3, 157.8, 153.7, 149.7, 148.1, 136.7, 124.4, 121.7, 107.2, 103.2, 95.7, 83.5, 80.0, 77.4, 77.3, 70.2, 69.6, 65.8, 58.1, 51.3, 51.1, 46.9, 44.6, 40.2, 38.6, 37.4, 28.2, 22.3, 21.1, 19.8, 18.0, 15.0, 14.5, 13.6, 13.5, 10.5; HRMS m/z calcd (M+H)⁺ for C₄₁H₅₆N₄O₁₁: 781.4018. Found: 781.4018.

### EXAMPLE 29

### Compound of Formula (I): D¹ is C≡C, Y¹ is isoxazol-3,5-diyl, A¹ is quinol-3-yl; X is fluoride; R¹ is hydrogen

### EXAMPLE 29A

### 3-(5-(tributylstannyl)-3-isoxazolyl)quinoline or

### Compound of Formula (iv) in Scheme 1: A¹ is quinol-3-yl; R is n-butyl

The desired product was prepared by substituting 3-quinolinecarbaldehyde oxime for 2-pyridinecarbaldehyde oxime in Example 3A.

### EXAMPLE 29B

### 3-(5-iodo-3-isoxazolyl)quinoline or

### Compound of Formula (v) in Scheme 1: A¹ is quinol-3-yl

The desired product was prepared by substituting Example 29A for Example 3A in Example 3B.

### EXAMPLE 29C

### Compound of Formula (I): D¹ is C≡C, Y¹ is isoxazol-3,5-diyl, A¹ is quinol-3-yl; X is fluoride; R¹ is C₆H₅C(O)

The desired product was prepared by substituting Examples 2A and 29B for Examples 1A and 3B, respectively, in Example 28A.

### EXAMPLE 29D

### Compound of Formula (I): D¹ is C≡C, Y¹ is isoxazol-3,5-diyl, A¹ is quinol-3-yl; X is fluoride; R¹ is hydrogen

The desired product was prepared by substituting Example 29C for Example 28A in Example 28B. MS m/z 849 (M+H)⁺; ¹³ C NMR (75 MHz, CDCl₃) δ 216.8, d (204.3 and 203.9), d (166.1 and 165.8), 106.4, 157.5, 154.0, 148.6, 148.4, 134.3, 130.5, 129.4, 128.4, 127.5, 127.3, 121.8, 106.3, 104.2, d (99.0 and 96.2), 96.6, 83.5, 80.7, 80.0, 78.7, 72.3, 70.3, 69.8, 65.8, 58.1, 50.7, 44.1, 40.5, 40.2, 38.3, 37.4, 28.1, d (25.4 and 25.1), 22.2, 21.2, 20.3, 17.6, 15.4, 13.7, 13.2, 10.6; HRMS m/z calcd (M+H)⁺ for C₄₅H₅₇FN₄O₁₁: 849.4081. Found: 849.4087.

It will be evident to one skilled in the art that the invention is not limited to the forgoing examples, and that it can be embodied in other specific forms without departing from the essential attributes thereof. Thus, it is desired that the examples be considered as illustrative and not restrictive, reference being made to the claims, and that all changes which come within the meaning and range of equivalency of the claims be embraced therein.

What is claimed is:

1. A compound of formula (I) 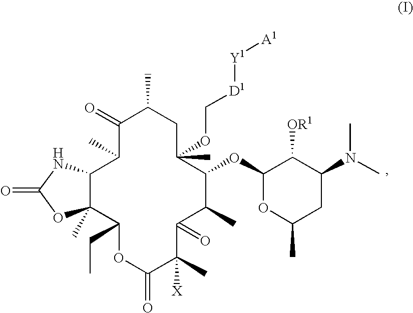
or a therapeutically acceptable salt or prodrug thereof, wherein
X is selected from hydrogen and fluoride;
D¹ is selected from CH═CH or C≡C;
Y¹ is selected from isoxazole, oxazole, isothiazole, dihydroisoxazole, and
dihydro-oxazole;
A¹ is selected from aryl and heteroaryl; and
R¹ is selected from hydrogen and R^{p} wherein R^{p} is a hydroxyl protecting group.

2. A compound according to claim 1 wherein X is hydrogen.

3. A compound according to claim 1 wherein X is fluoride.

4. A compound according to claim 1 wherein D is C≡C.

5. A compound according to claim 1 wherein Y¹ is isoxazol-3,5-diyl.

6. A compound according to claim 1 wherein A¹ is aryl.

7. A compound according to claim 1 wherein A¹ is heteroaryl.

8. A compound according to claim 1 wherein R¹ is hydrogen.

9. A compound according to claim 1 wherein R¹ is R^{p} and R^{p} is acetyl.

10. A compound according to claim 1 wherein R¹ is R^{p} and R^{p} is benzoyl.

11. A compound according to claim 1 of formula (Ia) 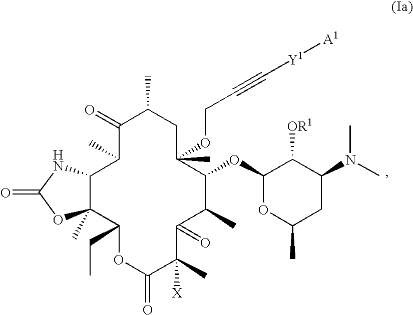
or a therapeutically acceptable salt or prodrug thereof, wherein
X is selected from hydrogen and fluoride;
Y¹ is selected from isoxazole, oxazole, isothiazole, dihydroisoxazole, and dihydro-oxazole;
A¹ is selected from aryl and heteroaryl; and
R¹ is selected from hydrogen and R^{p} wherein R^{p} is a hydroxyl protecting group.

12. A compound according to claim 11 wherein X is hydrogen.

13. A compound according to claim 11 wherein X is fluoride.

14. A compound according to claim 11 wherein Y¹ is isoxazol-3,5-diyl.

15. A compound according to claim 11 wherein A¹ is aryl.

16. A compound according to claim 11 wherein A¹ is heteroaryl.

17. A compound according to claim 1 of formula (Ib) 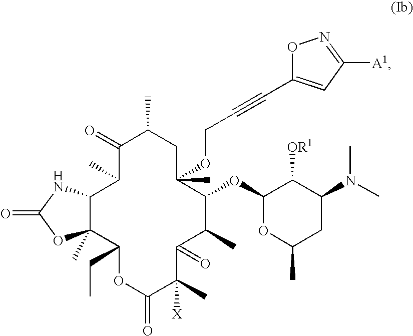
or a therapeutically acceptable salt or prodrug thereof, wherein
X is selected from hydrogen and fluoride;
A¹ is selected from aryl and heteroaryl; and
R¹ is selected from hydrogen and R^{p} wherein R^{p} is a hydroxyl protecting group.

18. A compound according to claim 17 wherein X is hydrogen.

19. A compound according to claim 17 wherein X is fluoride.

20. A compound according to claim 17 wherein A¹ is aryl.

21. A compound according to claim 17 wherein A¹ is heteroaryl.

22. A compound according to claim 1 of formula (Ii) 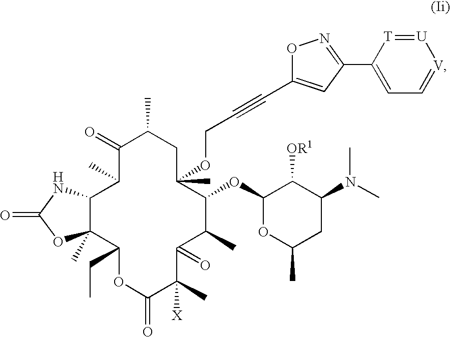
or a therapeutically acceptable salt or prodrug thereof, wherein
X is hydrogen or fluoride;
R¹ is selected from hydrogen and R^{p} wherein R^{p} is a hydroxyl protecting group; and
one of T, U, and V is nitrogen and the remainder are carbon.

23. A compound according to claim 22 wherein X is hydrogen.

24. A compound according to claim 22 wherein X is fluoride.

25. A compound according to claim 22 wherein T is nitrogen.

26. A compound according to claim 22 wherein U and V are each carbon.

27. A method for the preparation of a compound of formula (Ia) 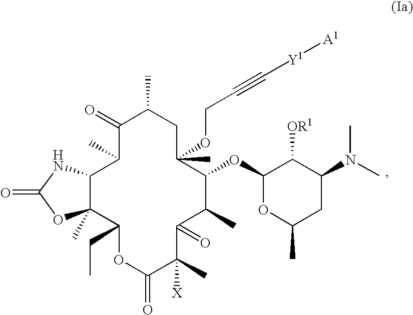
or a therapeutically acceptable salt or prodrug thereof, wherein
X is selected from hydrogen and fluoride;
Y¹ is selected from isoxazole, oxazole, and isothiazole;
A¹ is selected from aryl and heteroaryl; and
R¹ is selected from hydrogen and R^{p} wherein R^{p} is a hydroxyl protecting group, the method comprising:
(a) reacting a compound of formula (vi) 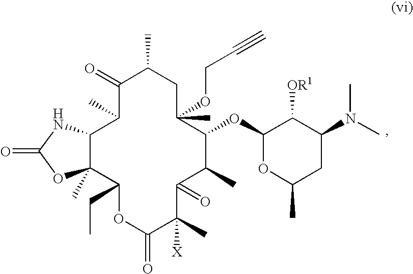
wherein
X is selected from hydrogen and fluoride, and
R¹ is selected from hydrogen and R^{p} wherein R^{p} is a hydroxyl protecting group,
with a compound of formula (vii)
I—Y¹-A (vii),
wherein
Y¹ is selected from isoxazole, oxazole, and isothiazole, and
A¹ is selected from aryl and heteroaryl,
a base, a coupling catalyst, and, optionally, an additive;
and
(b) optionally deprotecting the product of step (a).

27. A method for the preparation of a compound of formula (Ia) 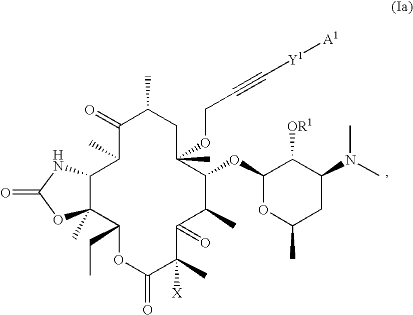
or a therapeutically acceptable salt or prodrug thereof, wherein
X is selected from hydrogen and fluoride;
Y¹ is selected from isoxazole, oxazole, and isothiazole;
A¹ is selected from aryl and heteroaryl; and
R¹ is selected from hydrogen and R^{p} wherein R^{p} is a hydroxyl protecting group, the method comprising:
(a) reacting a compound of formula (vi) 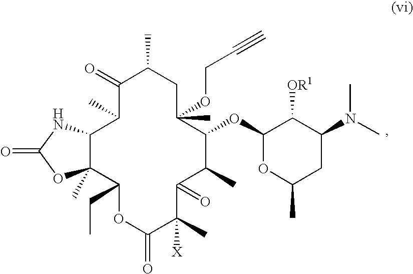
wherein
X is selected from hydrogen and fluoride, and
R¹ is selected from hydrogen and R^{p} wherein R^{p} is a hydroxyl protecting group,
with a compound of formula (vii)
I—Y¹-A¹ (vii),
wherein
Y¹ is selected from isoxazole, oxazole, and isothiazole, and
A¹ is selected from aryl and heteroaryl,
a base, a coupling catalyst, and, optionally, an additive;
and
(b) optionally deprotecting the product of step (a).

28. The method of claim 27 wherein the coupling catalyst is selected from dichlorobis(triphenylphosphine)palladium(II), tris(dibenzylideneacetone)dipalladium(0), tetrakis(triphenylphosphine)palladium(0), and dichlorobis(triphenylphosphine)nickel(II).

29. The method of claim 27 wherein the additive is selected from triphenylphosphine, triphenylarsine, copper(I) iodide, and mixtures thereof.

30. The method of claim 27 wherein the base is selected from triethylamine and N,N-diisopropylethylamine.

31. A method for the preparation of a compound of formula (Ib) 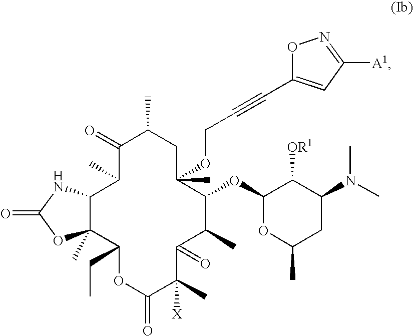
or a therapeutically acceptable salt or prodrug thereof, wherein
X is selected from hydrogen and fluoride;
A¹ is selected from aryl and heteroaryl; and
R¹ is selected from hydrogen and R^{p} wherein R^{p} is a hydroxyl protecting group, the method comprising:
(a) reacting a compound of formula (x) 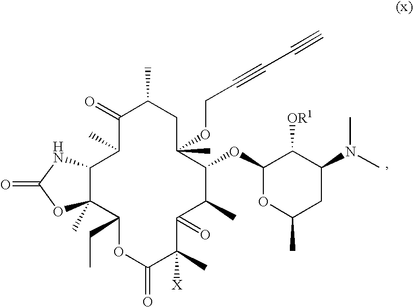
wherein
X is selected from hydrogen and fluoride, and
R¹ is selected from hydrogen and R^{p} wherein R^{p} is a hydroxyl protecting group,
with a compound of formula (ii) 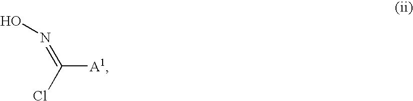
wherein
A is selected from aryl and heteroaryl,
and a base;
and
(b) optionally deprotecting the product of step (a).

32. The method of claim 31 wherein the base is selected from sodium bicarbonate, sodium carbonate, triethylamine, and N,N-diisopropylethylamine.

33. A composition comprising a compound according to claim 1 in combination with a therapeutically acceptable excipient.

34. A method of treating bacterial infections comprising administering a therapeutically effective amount of a compound having structural formula (I) 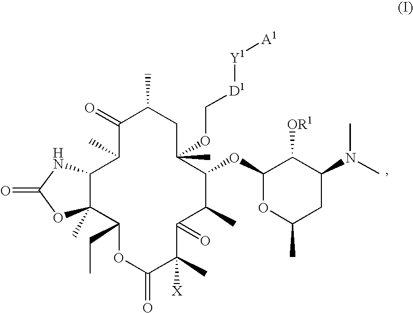
or therapeutically acceptable salt or prodrug thereof, wherein
X is selected from hydrogen and fluoride;
D¹ is selected from CH═CH or C≡C;
Y¹ is selected from isoxazole, oxazole, isothiazole, dihydroisoxazole, and dihydro-oxazole;
A¹ is selected from aryl and heteroaryl; and
R¹ is selected from hydrogen and R^{p} wherein R^{p} is a hydroxyl protecting group.

35. A compound selected from the group consisting of:
a compound of formula (I) wherein D¹ is C≡C, Y¹ is isoxazol-3,5-diyl, A¹ is pyrid-2-yl, X is fluoride, and R¹ is hydrogen;
a compound of formula (I) wherein D¹ is C≡C, Y¹ is isoxazol-3,5-diyl, A¹ is quinol-3-yl, X is hydrogen, and R¹ is hydrogen;
a compound of formula (I) wherein D¹ is C≡C, Y¹ is isoxazol-3,5-diyl, A¹ is quinol-2-yl, X is hydrogen, and R¹ is hydrogen;
a compound of formula (I) wherein D¹ is C≡C, Y¹ is isoxazol-3,5-diyl, A¹ is quinol-4-yl, X is hydrogen, and R¹ is hydrogen;
a compound of formula (I) wherein D¹ is C≡C, Y¹ is isoxazol-3,5-diyl, A¹ is 4-fluorophenyl, X is hydrogen, and R¹ is hydrogen;
a compound of formula (I) wherein D¹ is C≡C, Y¹ is isoxazol-3,5-diyl, A¹ is pyrid-4-yl, X is hydrogen, and R¹ is hydrogen;
a compound of formula (I) wherein D¹ is C≡C, Y¹ is isoxazol-3,5-diyl, A¹ is 4-cyanophenyl, X is hydrogen, and R¹ is hydrogen;
a compound of formula (I) wherein D¹ is C≡C, Y¹ is isoxazol-3,5-diyl, A¹ is pyrid-3-yl, X is hydrogen, and R¹ is hydrogen;
a compound of formula (I) wherein D is C≡C, Y¹ is isoxazol-3,5-diyl, A¹ is thien-2-yl, X is hydrogen, and R¹ is hydrogen;
a compound of formula (I) wherein D¹ is C≡C, Y¹ is isoxazol-3,5-diyl, A¹ is thiazol-2-yl, X is hydrogen, and R¹ is hydrogen;
a compound of formula (I) wherein D¹ is C≡C, Y¹ is isoxazol-3,5-diyl, A¹ is 3,4-difluorophenyl, X is hydrogen, and R¹ is hydrogen;
a compound of formula (I) wherein D¹ is C≡C, Y¹ is isoxazol-3,5-diyl, A¹ is 3-(trifluoromethyl)phenyl, X is hydrogen, and R¹ is hydrogen;
a compound of formula (I) wherein D¹ is C≡C, Y¹ is isoxazol-3,5-diyl, A¹ is 3,4-dichlorophenyl, X is hydrogen, and R¹ is hydrogen;
a compound of formula (I) wherein D¹ is C≡C, Y¹ is isoxazol-3,5-diyl, A¹ is 3-cyanophenyl, X is hydrogen, and R¹ is hydrogen;
a compound of formula (I) wherein D¹ is C≡C, Y¹ is isoxazol-3,5-diyl, A¹ is 4-cyano-3-(methylsulfanyl)pyrid-2-yl, X is hydrogen, and R¹ is hydrogen;
a compound of formula (I) wherein D¹ is C≡C, Y¹ is isoxazol-3,5-diyl, A¹ is thiazol-5-yl, X is hydrogen, and R¹ is hydrogen;
a compound of formula (I) wherein D¹ is C≡C, Y¹ is isoxazol-3,5-diyl, A is 6-chloroimidazo(2,1-b)thiazol-5-yl, X is fluoride, and R¹ is hydrogen;
a compound of formula (I) wherein D¹ is C≡C, Y¹ is isoxazol-3,5-diyl, A¹ is thiazol-5-yl, X is fluoride, and R¹ is hydrogen;
a compound of formula (I) wherein D¹ is C≡C, Y¹ is isoxazol-3,5-diyl, A¹ is thiazol-2-yl, X is fluoride, and R¹ is hydrogen;
a compound of formula (I) wherein D¹ is C≡C, Y¹ is isoxazol-3,5-diyl, A¹ is 3,4-dichlorophenyl, X is fluoride, and R¹ is hydrogen;
a compound of formula (I) wherein D¹ is C≡C, Y¹ is isoxazol-3,5-diyl, A¹ is pyrimidin-5-yl, X is fluoride, and R¹ is hydrogen;
a compound of formula (I) wherein D¹ is C≡C, Y¹ is isoxazol-3,5-diyl, A¹ is 2-methyl-2H-tetrazol-5-yl, X is hydrogen, and R¹ is hydrogen;
a compound of formula (I) wherein D¹ is C≡C, Y¹ is isoxazol-3,5-diyl, A¹ is 2-methyl-2H-tetrazol-5-yl; X is fluoride; and R¹ is hydrogen;
a compound of formula (I) wherein D¹ is C≡C, Y¹ is isoxazol-3,5-diyl, A¹ is 2-chloroquinol-3-yl, X is hydrogen, and R¹ is hydrogen;
a compound of formula (I) wherein D¹ is C≡C, Y¹ is isoxazol-3,5-diyl, A¹ is 3-methylbenzo(b)thien-2-yl, X is hydrogen, and R¹ is hydrogen;
a compound of formula (I) wherein D is C≡C, Y¹ is isoxazol-3,5-diyl, A¹ is pyrid-2-yl, X is hydrogen, and R¹ is hydrogen;
a compound of formula (I) wherein D¹ is C≡C, Y¹ is isoxazol-3,5-diyl, A¹ is quinol-3-yl, X is fluoride, and R¹ is hydrogen;
a compound of formula (x) wherein X is hydrogen and R¹ is hydrogen;
a compound of formula (x) wherein X is hydrogen and R¹ is R^{p} wherein R^{p} is acetyl;
a compound of formula (x) wherein X is hydrogen and R¹ is R^{p} wherein R^{p} is benzoyl;
a compound of formula (x) wherein X is fluoride and R¹ is hydrogen;
a compound of formula (x) wherein X is fluoride and R¹ is R^{p} wherein R^{p} is acetyl; and
a compound of formula (x) wherein X is fluoride and R¹ is R^{p} wherein R^{p} is benzoyl.

36. A compound according to claim 35 which is
the compound of formula (I) wherein D¹ is C≡C, Y¹ is isoxazol-3,5-diyl, A¹ is pyrid-2-yl, X is fluoride, and R¹ is hydrogen.

37. A method for treating bacterial infections comprising administering to a patient in need thereof a compound according to claim 36.

38. A pharmaceutical composition comprising a compound according to claim 36 in admixture with a pharmaceutically acceptable carrier.

39. A method for treating bacterial infections comprising the administration to a patient in need thereof a compound according to claim 17.

40. A pharmaceutical composition comprising a compound according to claim 17 in admixture with a pharmaceutically acceptable carrier.
